(19) United States
(12) Patent Application Publication
(10) Pub. No.: US 2019/0029235 A1
(43) Pub. Date: Jan. 31, 2019

(54) EFFICIENT, SCALABLE PATIENT-DERIVED XENOGRAFT SYSTEM BASED ON A CHICK CHORIOALLANTOIC MEMBRANE (CAM) IN VIVO MODEL

(71) Applicant: Baylor College of Medicine, Houston, TX (US)

(72) Inventors: Andrew G. Sikora, Houston, TX (US); Ravi Pathak, (US)

(21) Appl. No.: 15/773,988

(22) PCT Filed: Nov. 4, 2016
(86) PCT No.: PCT/US16/60664

§ 371 Date: Oct. 15, 2018

Related U.S. Application Data
(60) Provisional application No. 62/251,404, filed on Nov. 5, 2015.

(51) Int. Cl. A01K 67/027; G01N 33/50

(52) U.S. Cl. CPC A01K 67/0271; G01N 33/5088; A01K 2267/0331; A01K 2227/30; A01K 2207/12

(57) ABSTRACT

Embodiments of the disclosure encompass systems that utilize chick chorioallantoic membranes (CAM) as models for cancer xenografts, including at least patient-derived xenografts (CAM-PDX). In particular embodiments, the system employs the CAM-PDX to graft and culture different types of tumor tissue on a single or multiple eggs. In specific embodiments multiple tumor regions of a single tumor are cultured on a single egg. Frozen tissue is successfully revived, in specific embodiments. Downstream applications following successful establishment of CAM-PDX models are encompassed.

### CROSS REFERENCE TO RELATED APPLICATIONS

This application claims priority to U.S. Provisional Patent Application Ser. No. 62/251,404, filed Nov. 5, 2015, which is incorporated by reference herein in its entirety.

### TECHNICAL FIELD

Embodiments of the disclosure concern at least the fields of molecular biology, cell biology, medicine, life sciences research, cancer research, drug models, therapeutic testing, and so forth.

### BACKGROUND

Chorioallantoic membrane (CAM) assays have been used to study angiogenesis, tumor cell invasion and metastasis. The CAM model is useful because of its vascularity, which enhances the efficiency of tumor cell grafting and its high reproducibility. The half-life of at least certain test compounds is often longer in comparison to animal models, which facilitates analysis of potential anti-cancer compounds (for example) that are only available in limiting amounts. A CAM comprises a multilayer epithelium, including an ectoderm at the air interface, a mesoderm (or stroma), and an endoderm at the interface with the allantoic sac. A CAM also can include extracellular matrix (ECM) compounds, such as fibronectin, laminin, collagen type I and integrin αvβ3. The presence of these extracellular matrix proteins further enhances mimicry of the endogenous environment of cancer cells in a mammal.

Although the CAM assay is a well-established model for studying certain types of angiogenesis and metastasis in certain individual cancers, there is a long-felt need in the art for models that allow study of different types of cancer, serial passaging, intercompatability with other three-dimensional tumor culture systems, and so forth.

### BRIEF SUMMARY

Embodiments of the disclosure concern systems, methods, and compositions for testing and analysis of neoplastic matter (including cancerous matter or benign neoplasms) or non-cancerous matter, such as tumor tissue, in models that mimic an in vivo environment in which a tumor naturally resides. In particular embodiments, such systems, methods, and compositions concern the chick embryo chorioallantoic membrane (CAM) model for assaying tumor tissue. The tumor tissue in question may be of any kind, type, stage, origin, or grade of cancer; it may be primary patient-derived tissue or a tumor cell line. A single CAM model egg may harbor on its surface multiple types of tumors from multiple sources, in specific embodiments. The tumor tissue may be fresh or frozen.

The present disclosure establishes CAM-based patient-derived xenografts (PDX) as a model system for study of cancer biology and patient treatment response, including a scalable approach to capturing intratumoral heterogeneity (ITH). Although patient derived tumor xenografts (PDTX) are created when cancerous tissue from a patient's primary tumor is implanted directly into an immunodeficient mouse or into a xenopus model, the present disclosure utilizes a chick egg model.

In particular embodiments, a single CAM model is utilized for analysis of multiple tumor tissues at a given time. The multiple tumor tissues may derive from different locations of a single tumor in an individual or from different tumors of an individual, or a combination thereof. In specific embodiments, the different locations of tumor tissue on a single CAM model are encased or otherwise separated by a structure, such as a cast comprising an aperture for exposure to the CAM.

Prior to establishment of the CAM model, the tumor tissue to be utilized may have been cultured on another CAM model, may have come from another type of model (such as a mouse xenograft model, for example), including a patient-derived xenograft model, may have been obtained directly from an individual suffering from cancer or a benign neoplasm, or a combination thereof.

Following generation of the CAM model, the established tumor tissue in the model may be assayed in one or more methods for analysis of the tumor tissue. The established tumor tissue from the model may also be further transferred to other models, including other patient-derived xenograft models or other in vivo models, for example.

Embodiments of the disclosure include characterization of transcriptomic and epigenomic changes in cellular subpopulations of patient-derived and CAM-based PDX tissue. In some embodiments, baseline samples from geographically distinct tumor regions exhibit varying degrees of genomic, epigenomic, and proteomic diversity, which may be stably propagated forward across one or multiple CAM serial passages. In certain embodiments, geographically separated tumor regions have different relative compositions of cancer and stromal cell types, which may be maintained for one or a number of serial passages on CAM, for example.

In particular embodiments, the CAM model is enhanced to more closely mimic a natural tumor environment. In specific aspects, this is achieved at least in part by reconstituting the immune microenvironment of the tumor, and in particular aspects this occurs by providing the tumor in the CAM model an effective amount of any one or more types of immune cells, such as patient-derived immune cells, allogeneic immune cells, or cells engineered for anti-cancer therapy. The immune cells may be T cells, NK cells, NKT cells, B cells, and so forth.

In one embodiment, there is a method of establishing tumor tissue in a model, comprising the steps of: a) providing or obtaining one or more chick chorioallantoic membrane (CAM) egg models; b) providing or obtaining tissue from multiple regions of a mammalian tumor of an individual; and culturing the tissue from multiple regions of the tumor on separate locations of a single CAM model or on multiple CAM models; or providing or obtaining tissue from a patient-derived xenograft model or CAM model; and culturing the tissue on one or separate locations of a single CAM model or on multiple CAM models; or providing or obtaining tissue from multiple tumors of an individual; and culturing the tissue on separate locations of a single CAM model or on multiple CAM models. In specific embodiments, the method further comprises the step of: c) assaying the cultured tumor tissue. In some embodiments, the cultured tumor tissue is passaged to another model, including a tumor host model, such as a CAM model, a mouse model, a frog model, a dog model, guinea pig model, rat model. In specific embodiments the assaying comprises sequencing, gene expression profiling, tumor volume measurement, real-time imaging, or a combination thereof. In specific embodiments, the assaying comprises exposure of the cultured tumor tissue to a cancer therapy, such as a chemotherapy, radiotherapy, targeted agents, immunotherapy, or a combination thereof. In some embodiments, the tumor tissue is obtained from the individual prior to exposure to a cancer therapy, following exposure to a cancer therapy, or both or the tumor tissue is obtained from the individual prior to exposure to the cancer therapy, following exposure to the cancer therapy, or both. In some embodiments, an effective amount of the cancer therapy for the tumor tissue is determined. In certain embodiments, as a result of the method, the individual from which the tumor tissue was originally derived is provided a suitable cancer therapy.

In some cases, the culturing step comprises culturing the tissue within a physical barrier on the egg, wherein the barrier comprises an aperture allowing exposure of the tissue to the egg. In particular aspects, the barrier is ring-shaped. In some cases, the barrier is comprised of biologically inert material, such as silicon-based organic polymers, for example. In some cases, the mammal is a human or mouse. In some embodiments, the tissue is a mouse patient-derived xenograft model.

In particular embodiments, the step of providing or obtaining tissue to be grown on a patient-derived xenograft model or CAM model comprises providing or obtaining tissue from a patient-derived mouse xenograft model. In at least some cases, the cultured tissue is further provided to a model, such as an in vivo model; the model may be a patient-derived xenograft mouse model.

In some embodiments, cells from the cultured tissue are used for generating cell lines, and cells from the cultured tissue may be used for flow cytometry or viral transduction.

In specific embodiments, the obtained tissue may be subject to freezing temperatures prior to the culturing step. In particular aspects, cells from the cultured tissue are frozen.

In specific embodiments, the culturing steps utilize conditions suitable for three-dimensional tumor growth. In certain cases, cells from the cultured tissue are used for generating cell lines. Cells from the individual or from the patient-derived xenograft model or CAM model may be genetically engineered, such as genetically engineered with a viral or other targeted gene knockout/knock in delivery system; the cells may be genetically engineered to target a tumor suppressor or an oncogene. In specific embodiments, the culturing step comprises providing to the tissue one or more types of immune cells. The immune cells may be obtained from the individual or may be allogeneic to the individual.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 illustrates the overall concept of CAM-based PDX as applied to capturing intratumoral heterogeneity (ITH). Tumor samples from different geographic tumor regions are established on CAM, providing a “snapshot” of tumor diversity at the time of harvest. Serial passage and expansion of CAM PDX grafts provides material for assays.

FIGS. 2A, 2B, and 2C demonstrate gross and microscopic characteristics of CAM-based PDX. Representative example of F1 HNSCCA xenograft on CAM. 2A. Teflon ring on CAM, ready for grafting; 2B. Macroscopic appearance of tumor. 2C. Histologic appearance of tumor in background of CAM tissue.

FIGS. 3A and 3B show stability of gene expression profile between index tumor and CAM-PDX. 3A. Histological appearance of patient-derived breast cancer and F1 CAM-PDX line derived from that tumor. 3B. Scatter plot of differentially-expressed genes showing genes downregulated (red; above the diagonal line) and upregulated (green; below the diagonal line) in CAM with respect to index tumor. 3877/44,669 coding features (<10%) were >2-fold differentially expressed in patient and CAM-PDX tumors.

FIG. 4. Demonstrates a strategy for assessment of regional intratumoral heterogeneity (ITH) and stability of CAM-PDX lines. Multiple geographically distinct tumor regions are established independently on CAM and serially passaged. CAM-PDX and regional primary tumor fragments are analyzed by EDec and WES for determination of inter-tumoral, intra-tumoral and inter-PDX line heterogeneity, and stability of regional variation across PDX passages.

FIGS. 5A and 5B provide a representative in ovo MRI image of breast cancer PDX. 5A. MRI showing teflon ring, beginning of tumor nodule (arrow), and feeding vessels. 5B. Close up of 0.5 mm slice with tumor ROI identified for quantitative analysis. Peripheral feeding vessels are also visible.

FIG. 6 illustrates the CAM surrounding a young chick embryo. (reproduced from Marieb, Elaine Nicpon. Essentials of human anatomy and physiology. 5th ed. Menlo Park, Calif.: Benjamin/Cummings Pub. Co., 1997.)

FIGS. 7A, 7B, and 7C shows gross and microscopic characteristics of CAM-based breast PDX. 7A shows macroscopic appearance of CAM derived tumor. FIG. 7B shows comparison of mouse and CAM-PDX by histology (H&E). FIG. 7C shows evaluation of proliferative cells between mouse and CAM-PDX by Ki67 staining.

FIGS. 8A, 8B, 8C, and 8D demonstrate histological sections of CAM-PDX derived from different cancers. FIG. 8A shows histology of patient derived CAM-PDX breast tumors. FIG. 8B shows histology of patient-derived adnexal CAM-PDX. FIG. 8C shows H&E section of patient derived skin squamous cell carcinoma. FIG. 8D shows H&E section of patient derived oral squamous cell carcinoma.

FIG. 9 shows serial passage of breast PDX across multiple generations. WHIM-12 breast cancer derived from mouse PDX was successfully passaged across multiple generations while maintaining morphological stability and growth potential.

FIGS. 10A-10B demonstrate CAM-PDX established from cryopreserved tumor specimens. FIG. 10A provides Ki67 staining showing actively proliferating cells in frozen WHIM12 tumors revived on CAM (FIG. 10B). WHIM-12 breast cancer derived from mouse PDX and cryopreserved for three months was successfully revived on the CAM.

FIGS. 11A, 11B, 11C, and 11D demonstrate stability of gene expression profile between index tumor and CAM-PDX. FIG. 11A shows histological appearance of patient-derived breast cancer and F1 CAM-PDX line. FIG. 11B demonstrates scatter plot and FIG. 11C shows heat map of differentially-expressed genes showing genes downregulated (red; above the diagonal line) and upregulated (green; below the diagonal line) in CAM with respect to index tumor. 2709/44,669 annotated coding features (6%) were >2-fold differentially expressed in patient and CAM. FIG. 11D shows canonical pathways altered in the top 100 upregulated genes. The majority of the top pathways from which genes are differentially regulated belong to immune response. This is consistent with the transition of tumor to a different host (Human to Chicken in case of the CAM-PDX).

FIGS. 12A-12F show a horizontal method of preparing CAM eggs. 12A: Identification of embryo and vasculature using the candler, 12B: Making a hole in the shell, 12C: Applying suction to the hole at the naturally-occurring air sac, 12D: Visualizing the air bubble showing successful dropping of the CAM away from the shell, 12E-12F: Opening a window in the shell using the Dremel rotary tool. From Li, M., Pathak, R. R., Lopez-Rivera, E., Friedman, S. L., Aguirre-Ghiso, J. A., Sikora, A. G. “The In Ovo Chick Chorioallantoic Membrane (CAM) Assay as an Efficient Xenograft Model of Hepatocellular Carcinoma.” JoVE. 2015 Oct. 9 (104).

FIGS. 13A and 13B demonstrate a novel vertical method of preparing CAM eggs. 13A: Internal arrangement of the egg with the inner membrane (grey) associated with the CAM (red). 13B: Method of separating the inner membrane from the CAM by accessing the layer through the window cut at the air sac end of the egg.

FIG. 14 shows breast PDX serially passaged across multiple generations. Serial passage of WHIM 12 breast PDX passaged across multiple generations on the egg. The red circle within the egg highlights the tumor. F0-F6 indicates increasing generations for the passaged tumors.

FIGS. 15A-15C demonstrate shuttling patient tumors via egg before grafting to mouse models. 15A: Patient with tumor (highlighted in red). 15B: Resected patient tumor (in red) grafted on the egg. 15C: Egg-derived patient xenograft (in red) grafted in mouse.

FIG. 16A-16G shows drug sensitivity testing of mouse derived PDX in eggs. 16A: Macroscopic appearance of CAM derived tumor. 16B: Comparison of mouse and CAM PDX by histology (H&E). 16C: Evaluation of proliferative cells between mouse and CAM derived PDX by Ki67 staining. 16D: Patient with tumor (highlighted in red). 16E: Resected patient tumor (in red) grafted in mouse. 16F: Mouse-derived patient xenograft (in red) grafted in eggs. 6G: Different drugs tested on egg-derived patient xenografts. Eggs without tumors appear to be responsive to the drug treatment.

FIGS. 17A-17D demonstrate derivation of primary cell lines from egg-derived PDX. 17A: Patient with tumor (highlighted in red). 17B: Resected patient tumor (in red) grafted on the egg. 17C: Tumor cells obtained from the egg-derived patient xenografts. 17D: Tumor cells cultured on a petridish using appropriate conditioning growth medium.

FIGS. 18A-18C show converting of 2D head and neck cancer cell line to 3D. 18A: Head and neck cancer cell line SCC-90 converted into vascularized 3D tumor (indicated with an arrow within the white ring) on the egg. 18B and 18C: Histological (H&E) assessment of 3D tumors showing tumor cells indicated by white arrows.

FIGS. 19A-19C illustrate a proposed automation platform “OvoScreen”. 19A: Cell/tumor suspension injected in the eggs using automated syringes. The syringes can also be used to deliver drugs, small molecules and other agents in eggs. 19B: Eggs with 3D tumors (in red). 19C: Light source to detect tumor cells that are labeled with appropriate fluorescent dyes.

FIGS. 20A-20B show head and neck cancer tumors treated with Adenovirus. 20A: Histology of control 3D tumors derived from Head and Neck cancer cell line SCC47. 20B: Histology of 3D tumors derived from Head and Neck cancer cell line SCC47 and treated with oncolytic adenovirus. The treatment shows antitumor effect.

FIGS. 21A-21D demonstrate stability of gene expression profile between index tumor and CAM-PDX. 21A: Histological appearance of patient-derived breast cancer and F1 CAM-PDX line. 21B: Scatter plot and 21C: Heat map of differentially-expressed genes showing genes down-regulated (red) and up-regulated (green) in CAM with respect to index tumor. 2709/44,669 annotated coding features (6%) were >2-fold differentially expressed in patient and CAM. 21D: Canonical pathways altered in top 100 up-regulated genes.

FIGS. 22A-22B show representative in ovo MRI image of breast cancer PDX. 22A: MRI showing teflon ring, beginning of tumor nodule (arrow), and feeding vessels. 22B: Close up of 0.5 mm slice with tumor ROI identified for quantitative analysis. Peripheral feeding vessels are also visible.

### DETAILED DESCRIPTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

### I. General Embodiments

The current disclosure describes a novel method for establishing patient derived xenografts (PDX) in fertilized eggs (such as chicken eggs) by implanting patient tumor material on the chorioallantoic membrane (CAM) of the egg. Suitable tumor material for the process includes, but is not limited to, surgical or biopsy specimens performed as part of standard-of-care treatment, for example. The subject of the disclosure harnesses the naturally occurring egg CAM's ability to serve as a nutrient membrane in order to support the growth of tissue, such as growth of an individual's tumor explant. The novel method utilizes this ability of the CAM to maintain much of the unique genomic, cellular, and molecular characteristics of the original patient tumor. Using CAM-grown PDX, described herein are a variety of applications for the method, such as for a process to predict the sensitivity of a tumor to chemotherapy, radiotherapy, targeted agents, and/or other therapeutic agents for purposes of research or for personalized cancer therapy for an individual in need thereof. The methods described herein can also be applied to generate a renewable source of patient tissue for various assays by repetitive subculture of the CAM-grown PDX, for example onto new eggs, in certain embodiments.

### II. Embodiments of the CAM Model

The chorioallantoic membrane (also referred to as the chorioallantois or CAM) comprises a vascular membrane located in the eggs of some amniotes, for example birds and reptiles. The membrane is formed by the fusion of the mesodermal layers of the allantois and the chorion. The CAM comprises the following three different layers: the chorionic epithelium, the mesenchyme and the allantoic epithelium.

Embodiments of the disclosure include CAM models in which desired tumor tissue is established on one or more fertilized chick eggs. In specific embodiments, the tumor tissue is from an individual that is known to have cancer or the tissue is tissue that is suspected of being cancerous.

In some cases, the CAM model may be generated by horizontally-configured means. Generally, fertilized chicken eggs (for example, 6-, 7-, 8-, 9-, or 10-day old) are incubated in a humidified 37° C. chamber. Under sterile conditions, the eggshell surface is cleaned, and a window is created at the air sac end. Cells may be combined with a basement membrane/extracellular matrix extract (such as a natural or synthetic hydrogel (including at least collagen type 1 as a natural hydrogel or PEG as a synthetic hydrogel), laminin, fibrin, hyaluronic acid, chitosan, Matrigel®, or a combination thereof. Cells (including cells with the basement membrane/extracellular matrix extract, where appropriate) are then implanted and allowed for a period of time to graft (for example, two days). In some embodiments, the cells are implanted within a Teflon ring. Prior to implantation, the cells are comprised within a suitable buffer, such as PBS that may be supplemented with certain salts. The CAMs may (such as calcium and magnesium) be accessed through the window on certain time periods following engraftment for desired treatments and may be harvested following the treatments, or they may be harvested prior to treatment or another application that occurs elsewhere. Tumor growth and characteristics may be evaluated using standard techniques, such as H&E staining for subcellular structures or terminal deoxynucleotidyl transferase dUTP nick end labeling (TUNEL) staining, for example. Examples of applications for the CAM model are described in Section III below.

In specific embodiments, CAM xenografting details are provided, although the skilled artisan recognizes that such parameters may be optimized by routine methods in the art. For example, tumor specimens derived from patient- and mouse PDX-derived tumors may be cut into 100-200-mg, and incubated in minimal essential medium (MEM) supplemented with antibiotics for 15-30 minutes. The morcelized tumor pieces may be placed in a suspension of PBS (containing calcium and magnesium) and Matrigel®. The ratio of PBS to Matrigel® is optimized at 1:1, in specific embodiments. The morcelized mix is then explanted onto the vascularized CAM of 6-7 day chick embryos. Explants may be incubated at 37° C. with 60-70% humidity. At day 17, for example, chicks may be humanely euthanized and tumors processed for downstream applications.

In embodiments where there is cryopreservation of CAM-PDX tumors, the following regimen may be employed. Tumors grown on CAM may carefully be separated from the underlying CAM membrane and Matrigel®. Tumors may be washed in PBS (—calcium and magnesium), and incubated immediately into DMEM media containing 10% FBS for 5-10 minutes. The tumors may be subsequently transferred into a cryovial containing a freezing mixture of DMEM media with 10% FBS and 10% DMSO. The cryovials may be frozen in a step-wise manner at −80° C. for 24-48 hours, and shifted to liquid nitrogen storage tanks for long-term storage.

In certain embodiments, one may utilize frozen tissue for establishment of CAM tumors, including CAM-PDX tumors. An example of a procedure for frozen tissue follows, and in specific embodiments it is employed for re-derivation of viably frozen PDX tissue. Tissue is recovered from a frozen storage medium (such as from liquid nitrogen) and thawed immediately on ice. The freezing media is removed from the tube and 1 mL high glucose DMEM is added to the storage vessel (such as a tube). The tumor material is suspended in the fresh media and mixed well before being transferred into a 15 ml conical tube containing 14 ml of high glucose DMEM. The tumor is washed thoroughly in the tube by repeated pipetting, and the media is discarded. The process is repeated 1-2 times with 15 mL of high glucose DMEM. After the final wash, another 15 mL of high glucose DMEM is added and the tumor is placed on ice, for example if transplanting immediately. Before grafting onto the CAM, the tumor is washed thoroughly (2-3 times) in PBS containing calcium and magnesium to remove all traces of media.

In particular embodiments of the disclosure, the model employs a physical barrier to prevent tumor tissue from growing outside the barrier and yet have access to the egg. Thus, in specific embodiments the physical barrier comprises an aperture so that tumor tissue can be in contact with at least part of the egg. In this manner, multiple tumors may be established on a single CAM model egg and the tissue from the different tumors will not grow together. The physical barrier may be of any kind so long as tissue cannot grow beyond the barrier. In specific embodiments, the barrier is a cast or mold. The barrier may be of any material so long as it is biologically inert, and it may be of any suitable shape. In specific embodiments, the barrier is ring-shaped. In particular aspects, the barrier is comprised of at least one silicon-based organic polymer, such as polysiloxanes, or fluoropolymer. A specific example of a barrier material includes polytetrafluoroethylene (Teflon®). When more than one barrier is employed on a single or multiple CAM eggs, the different barriers may be distinguished by one or more types of markings, such as having different colors, numbers, and/or letters incorporated into the individual barriers, for example. In certain embodiments, one achieves sufficient isolation of individual tumors for targeted drug delivery, for example.

In certain embodiments, the CAM model is manipulated to enhance development of three-dimensional (3-D) tissue growth. Although the parameters may be optimized using routine methods in the art, one may perform the following to facilitate 3-D growth: a) preparing the eggs with a suitable amount of vasculature without rupturing any vessels; b) using an optimized PBS:Matrigel ratio; and/or c) maintaining a high humidity (at least about 80% during the entire engraftment period, for example).

Although the chicken egg is one embodiment of egg for the CAM model, in other embodiments other types of eggs are utilized. The egg for the model is preferably sufficiently vascularized, including avian or reptilian eggs that have a highly vascularized chorioallantoic membrane supporting the growth of the embryo. In specific embodiments, the bird is a chicken, turkey, duck, goose, quail, pheasant, grouse, ostrich, emu, cassowary or kiwi. A mixture of types of eggs may be used for the same application, in some cases.

### III. Embodiments of Uses for the CAM Model

The present disclosure utilizes an inexpensive and efficient system for analyzing tissue. The tissue may be of any kind, but in specific embodiments the tissue comprises neoplastic cells, including in the form of tumor tissue. The tissue may be known to comprise cancer cells or suspected of comprising cancer cells. Tumor source material includes patient and mouse PDX derived tumors, in addition to different cancer cell lines.

Embodiments of the disclosure allow establishment of tissue in a model system and, in particular embodiments allows subsequent processing, storage, and/or analysis of tissue taken from the established model system. The system allows analyzing of different types of cancer tissue, including cancers having different tissue of primary origin, and cancers that are any kind, type, stage, origin, or grade of cancer. The cancer may be lung, breast, colon, prostate, pancreatic, ovarian, skin, liver, kidney, spleen, thyroid, stomach, head and neck (laryngeal, oral cavity, oropharyngeal, hypopharyngeal, nasopharyngeal squamous cell carcinomas or other histologies), adnexal, cervical, adrenal gland, pituitary gland, gall bladder, metastatic carcinoma of unknown primary site and so forth. The models may be grafted with primary cancer tissue or metastatic cancer tissue, and in particular embodiments invasion and metastasis of the cancer tissue is analyzed in the system. In specific embodiments, the model utilizes tumor tissue derived from immortalized cell lines. Such tumor tissue, following their establishment in the CAM models, are subsequently passaged and may be grown in their three-dimensional (3-D) forms.

[In particular embodiments, cells cultured in a CAM model are cultured into three-dimensional (3-D) tumors that can be serially grafted or that can be maintained in their 3-D form, for example. This is a significant advantage for the methods of this disclosure, because 2-D cell culture models seldom or never recapitulate the 3-D tumor microenvironment, and most skilled artisans otherwise have to use expensive mouse PDX models. In specific embodiments, the cells are recombinantly modified. Examples of manipulation include making stable cell cancer cell lines using viral (for example, lentiviral) and other targeted gene knockout/knock in delivery systems. In specific examples, such manipulation for the cells targets tumor suppressor, oncogenes and other targets that are or might be targets for therapies.

In certain embodiments, the CAM models encompassed by the disclosure employ tumor tissue from a mammal, and in some cases that mammal may require therapy for cancer. In such cases, the analysis of the tumor tissue from the mammal allows determination of one or more specific therapies for the individual. In other embodiments, the CAM model employs tumor tissue from an individual that will not necessarily be given a therapy based on the analysis of the CAM model (such as tumor tissue donated for research purposes).

Following establishment of the CAM models of the disclosure, the engrafted and growing tumor tissue may be analyzed in the model; or part of the tumor graft may be extracted from the model and subject to analysis; or part of the tumor graft may be processed for storing, such as storage that includes freezing, or for deposit in a tumor bank; and/or part of the tumor graft may be used at least in part for the basis for establishing a graft on another model, such as another CAM model or a mouse model.

In certain aspects, CAM-PDX is a versatile and scalable approach to generating patient-derived xenografts, including for cancer tissue bio-banks, ex vivo cancer models, and as a screening platform for precision cancer medicine, for example.

The following description addresses multiple applications for analysis of the tumor tissue from a CAM model of the disclosure:

Analysis of Heterogeneity of Tumors

Development of resistance to therapy is common in cancer, and one important cause of treatment resistance occurs when different regions of the same tumor differ in their sensitivity to therapeutic approaches and thus are able to resist therapy. Embodiments of the disclosure provide CAM models that can analyze treatment sensitivities that differ across different locations in a patient's tumor, because one or more CAM models may provide for growth of multiple regions from the same tumor. Such information allows one to select optimal therapeutic approaches that decrease the chance of developing treatment resistance, thus leading to more durable responses and improved survival. Thus, there is in the disclosure determination of the sensitivity of tumors to treatment capable of overcoming the hurdle posed by regional differences in tumor treatment response.

Therefore, in specific embodiments of the present disclosure, there is improvement of patient outcomes through development of a completely novel approach to capturing the different genetic and drug sensitivity profiles that can occur in different regions of a patient's tumor. The CAM models allow for direct assessment of a tumor's susceptibility to different drugs, in certain embodiments. In specific embodiments, one can divide tumors into different regions and grow samples from each of these regions independently in the same or different chicken eggs, thus taking a “snapshot” of the regional differences seen in a patient's tumor that can be used for comprehensive testing of sensitivity to different therapies. In some embodiments of the disclosure, regional differences in tumor characteristics can be successfully captured by growing samples from multiple tumor regions of a single tumor or samples from multiple tumors on one or more eggs (for example, to simultaneously culture the primary tumor and a regional or distant metastasis prior to doing comparative studies (such as various genomic or epigenomic or transcriptomic assays, drug sensitivity, etc.). The efficiency and scalability of the CAM-PDX model of the disclosure facilitates capture of intratumoral heterogeneity, because it would be practical in the system to sample multiple tumor regions and establish different CAM-PDX from each, for example.

Serial Passaging Between Models

Particular embodiments allow for methods for serial passaging as part of processing of CAM tumors (including PDX tumors) for assays. Establishment of such methods includes demonstration of the degree and nature of baseline ITH in primary tumors, determination of the concordance of baseline, and analysis of the profiles of the tumors of subsequent passages.

In a specific and solely illustrative aspect, there is the following example: demonstration of the degree and nature of baseline ITH in primary HNSCC and breast tumors by whole exome sequencing (WES); epigenomic analysis of methylated DNA; transcriptomic analysis of gene expression; proteomic analysis of protein expression; and so forth.

In particular embodiments, tissue from the CAM model is passaged over one or more generations. The tissue may then be immediately analyzed, it may be deposited in a tumor bank, it may be cryopreserved, or it may be established in another model of any type, including another CAM model, an in vivo mouse model (including an in vivo mouse PDX model), and so forth. In particular embodiments, the CAM model serves as a viable host for generating xenografts for tumors obtained from mouse PDX models (PDX-Chicken), and tumors obtained directly from patients (Patient-Chicken). In specific embodiments, one can generate mouse PDX from chicken derived tumors that are of human origin (Patient-Chicken-Mouse PDX). In some cases, the model comprises a patient-mouse-chicken PDX, and such a model may be used for drug testing in certain aspects.

Drug Testing

In a particular embodiment one can employ CAM models, including CAM-based PDX models, for drug sensitivity testing. In specific aspects, one can optimize drug sensitivity assays on CAM and perform dose range-finding studies. In specific aspects, one can perform in ovo drug sensitivity testing of PDX lines, for example. The drug may be of any type, including chemotherapy, hormone therapy, immunotherapy, steroids, tyrosine kinase inhibitors and other targeted agents, bisphosphonates, and so on. The drug may be an alkylating agent; antimetabolite; anti-tumor antibiotic; topoisomerase inhibitor; mitotic inhibitor; corticosteroid; and so forth. In cases wherein the therapy is immunotherapy, the composition may be patient-derived immune cells, allogeneic immune cells, engineered immune cells (chimeric antigen receptor (CAR) T cells, for example), and so forth.

Revival of Frozen Tumor Material

In particular embodiments, cancer material that is frozen or has previously been frozen may be processed for use in a CAM model of the disclosure. Such material may be obtained as a repository for a particular individual or may come from a general tumor bank, for example. The tissue may be normal tissue from an individual that is housed in a repository and then utilized in a CAM model for comparison to tumor tissue that has subsequently developed in the individual.

In specific embodiments, one can successfully revive frozen tumor material from different cancer types stored at −80° C. Multiple xenografts have been established with consistently high take rates (70-80%). Frozen material includes freshly frozen tumors derived from patient, or mouse and egg derived PDX models, for example.

Imaging Methods for Tumor Assessment

The CAM based model allows one to image the tumors or image explants from the tumors. In specific embodiments, one can image tumors in real time. In particular aspects, there is measurement of tumor volumes and other parameters, for example using an MRI based imaging method that can perform real time imaging of tumors in a short time.

Deriving Primary Cell Lines

In particular embodiments, one can establish CAM models using tumor tissue of interest, and grafted, established tissue from the model may be utilized for cell culture. Patient derived tumors have been successfully grafted on a CAM, and the xenografts have been used to derive primary cell lines, in certain embodiments. The tumor cells derived from the xenografts have been used for multiple applications, such as flow cytometry and viral transduction, for example. In cases where tumor material is limiting, the CAM based method can generate additional tumor material from the source, thereby facilitating downstream applications.

Genomic Profiling

In particular embodiments, CAM models of the disclosure are utilized for genomic profiling of one or more regions of a tumor. Engraftment and serial passage on CAM may be a strategy for obtaining sufficient tumor material to run one or more genomic, epigenomic, transcriptomic, or proteomic tests. A xenograft model was generated for a patient derived tumor (breast cancer), and its detailed genomic profile was analyzed using a microarray, such as Affymetrix Gene Chip arrays. There was a very high degree of correlation between the patient and the CAM derived tumors, thereby establishing for the models the utility for drug sensitivity and efficacy assays. The entire process was completed within 2-3 weeks as compared to the existing mouse models that can take months.

### EXAMPLES

The following examples are presented in order to more fully illustrate the preferred embodiments of the invention. They should in no way, however, be construed as limiting the broad scope of the invention.

### Example 1

### A Rapid and Efficient Chick-Based Strategy for Cancer Xenografts

Embodiments of the disclosure provide a fast, cost-effective, and reproducible avian xenograft model that exploits the chorioallantoic membrane (CAM) for cancer xenografts.

Introduction:

Patient-derived xenograft (PDX) mouse models widely used in cancer research have contributed immensely to our understanding of cancer biology. However, despite being the current standard for PDX studies, there are a number of factors that limit the use of these models. Maintaining these PDX mouse models is laborious, time consuming and expensive. Additionally, xenografts in nude mice have displayed variable viability post implantation with engraftments rates ranging from 25-75% depending on the tumor type. This, combined with long experiment turn-around time (months to years), limits the reproducibility and degree to which the PDX mouse can be scaled.

CAM that surrounds and nourishes the developing chick embryo is immunodeficient and highly vascularized, properties that have been exploited herein to generate a natural in vivo model capable of supporting tumor growth, angiogenesis, and even metastasis. The present disclosure provides a fast, cost-effective, and reproducible avian xenograft model that exploits the chorioallantoic membrane (CAM) for cancer xenografts.

Exemplary Methods:

Tumor specimens (100-200-mg) are incubated in minimal essential medium (MEM) supplemented with antibiotics for 60-90 minutes. Tumor fragments (intact pieces or tumor mush) are mixed in a suspension of PBS and Matrigel®, and subsequently explanted onto the vascularized CAM of 6-day chick embryos, followed by incubation at 37° C. with 60-70% humidity. At day 17, chicks are euthanized via hypothermia (incubation on ice for 1 hour). Tumor explants and the surrounding CAM are assessed for viability, both grossly and microscopically.

Results:

Three-dimensional, vascularized tumors were successfully grown using tumor specimens from breast cancers (mouse PDX derived), skin cancer, oral squamous carcinoma and adenexal carcinomas derived from patient resections. The take rates for the tumor xenografts were between 60-75% for different tumor types, and once established showed high survival rates (>90%) for all xenografts. The tumors cells grown on CAM histologically resemble the original tumors, with actively proliferating regions within the xenografts.

Discussion:

The results successfully demonstrate the efficiency and reproducibility of the chick-based model across multiple tumor types. Furthermore, the model offers a unique advantage of providing easy access to the CAM and the tumor graft/plaque, which can be exploited to administer various drugs and anti-cancer compounds for efficacy testing, for example. In specific embodiments, the model is useful as a tool for maintaining cancer tissue bio-banks and as a screening platform for multiple drugs/compounds, for example.

### Example 2

### CAM-PDX Systems for Analyzing Intratumoral Heterogeneity

The present disclosure provides a simple and inexpensive xenograft system capable of efficiently engrafting and culturing multiple tumor samples that greatly increases the ability of PDX to survey intratumoral heterogeneity, for example, and enhance the predictive power of personalized cancer therapy approaches.

As described herein, chick chorioallantoic membrane (CAM)-based PDX is an efficient, scalable approach to capturing intratumoral heterogeneity and that in specific embodiments may be utilized for personalized cancer therapy. The chicken egg is a robust, self-contained, and inexpensive bioreactor capable of supporting growth of implanted cancer cell lines and tumor tissue (Petruzzelli et al., 1993). Described herein are a successfully grown, wide variety of primary patient-derived tumors on CAM, including head and neck squamous cell carcinoma (HNSCC) and breast tumors, for example. In specific embodiments, one can use CAM-based PDX as a tool for capturing intratumoral heterogeneity to more comprehensively determine tumor drug sensitivities and likelihood of therapeutic failure due to treatment resistance, for example.

One can generate a “snapshot” of intratumoral heterogeneity with CAM-based PDX by collecting tumor samples drawn from multiple geographic locations across a series of primary HNSCC tumors, for example. A portion of each sample may be used to determine baseline genomic/epigenomic profiles, and the remainder may be used to establish CAM-based PDX lines. Heterogeneity may be assessed across multiple dimensions of tumor phenotype including whole-exome sequencing (WES); and epigenomic deconvolution (EDec). EDec is a two-stage computational method that makes use of cell-type marker loci inferred from reference epigenomes (Amin et al., 2015) to determine average methylation profiles, average gene expression profiles, and relative proportions of each constituent cell type in the sample. This method is uniquely suited to analyze PDX stability and diversity because it profiles not only epigenomic and transcriptional changes, but allows quantitative assessment of the relative contributions of tumor, stromal, and immune cells making up the tumor tissue. For each dimension of assessment, the degree of initial intratumoral diversity is determined, and stability of these profiles is assessed across serial CAM passages. In specific embodiments, one can capture intratumoral heterogeneity with CAM-based PDX and preserve this diversity across multiple tumor passages.

CAM xenografts closely approximate in vivo drug sensitivity obtained in mouse xenograft models even in situations where the identical cell line grown in monolayer on plastic does not (Lopez-Rivera et al., 2014). One can demonstrate feasibility of drug sensitivity testing with CAM-based PDX. Mouse PDX lines with known drug sensitivity profiles may be transferred to the CAM. Once established on CAM, the tumors are treated with a panel of chemotherapeutic and targeted therapy agents, tumor proliferation and viability is measured, and dose-responses are established. The relative sensitivities of tumors growing on CAM to therapeutic agents are compared to their previously-determined sensitivity profiles obtained as mouse xenografts. In specific embodiments, the ability of CAM-based PDX to serve as an accurate predictor of tumor sensitivity to therapeutic agents is demonstrated. In certain embodiments, such demonstration lays the foundation for further studies comparing an ovo drug response to patient clinical responses.

Embodiments of the unique PDX model system as a scalable and cost-effective approach to incorporating assessment of intratumoral heterogeneity into personalized cancer medicine are provided herein.

### Example 3

### CAM-PDX Systems as Cancer Biology Models

“Avatar”-Based Approaches to Precision Cancer Medicine.

Precision, or “personalized,” cancer medicine focuses on using individual tumor and/or host characteristics to select optimal patient-specific therapeutic regimens. Strategies may be focused on identification of genomic, epigenomic, or multi-dimensional “pan-omic” profiles previously shown to be associated with treatment response or resistance; or on assessment of treatment resistance/sensitivity profiles based on direct interrogation of tumor phenotype. An example of this latter approach is patient derived tumor xenografts (PDX) grown in immunodeficient recipient mice. Once established in so-called “mouse avatars,” PDX can be serially passaged, expanded, and used for direct testing of in vivo drug sensitivity (Malaney et al., 2014). Many groups working in mouse-based PDX models have demonstrated genomic and phenotypic concordance between the initial patient tumor and PDX in mouse; stability of these profiles across multiple generations of serial passage; and correlation of treatment sensitivity profiles between patient and PDX models. Thus, PDX avatars represent a very important step towards the ultimate goals of precision medicine: to accurately predict the response to therapy of an individual patient's tumor, and use this information to select the treatment approach with highest chance of success.
Intratumoral Heterogeneity (ITH).

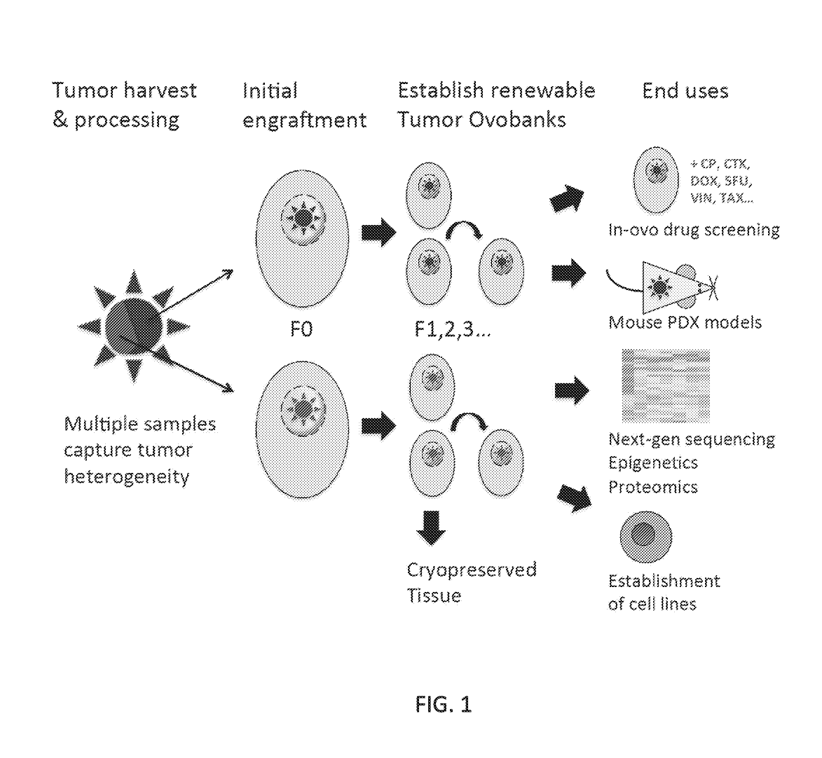

While the profound differences between histologically “identical” tumors in different patients provide the rationale for precision medicine, genomic, epigenomic, and transcriptomic differences between different geographical regions of a single tumor can also be profound (Pribluda et al., 2015). An emerging literature describes often striking genome- and transcriptome-level differences among spatially distinct tumor regions that can provide a mechanism for treatment resistance and post-treatment persistence of minimal residual disease (Fedele et al., 2014; Somasundaram et al., 2012). Thus, regional ITH poses an obvious barrier to precision cancer medicine (Pribluda et al., 2015; Zhang et al., 2013) and a rationale for developing PDX models that can efficiently capture tumor fragments from multiple geographically distinct tumor regions. In the present disclosure, ITH may be analyzed in CAM-based PDX, with a variety of end uses (FIG. 1).
The Chick Chorioallantoic Membrane (CAM) as a Model for Studying In Vivo Cancer Biology.

The scientific utility of the chicken egg as a self-contained in vivo model for cancer research was realized as early as 1913 when the first primary human tumor was engrafted onto the CAM (Murphy, 1913). The highly vascularized CAM supports and nourishes the developing embryo, and can similarly support engraftment and in vivo growth of both primary tumors and cancer cell lines until approximately day 18 when the developing immune system will reject the xenograft. Both primary tumors and cell lines form 3-D, vascularized tumors that maintain many of the properties of cancer cells growing in vivo that are often lost in 2-D tissue culture, making CAM models ideal for study of cancer cell characteristics such as angiogenesis, invasion, and even metastasis of tumor cells into the developing chick (Deryugina et al., 2008; Lopez-Rivera et al., 2014; Ribatti, 2014).
The CAM as a Robust and Efficient Patient-Derived Xenograft (PDX) Model.

The present inventors have successfully established 50 CAM PDX lines derived from 8 patients with 5 different tumor types, including head and neck squamous cell carcinoma (HNSCC), breast cancer, adnexal carcinoma, papillary thyroid cancer and skin squamous cell carcinoma. The overall take rate of 80% compares favorably to that in mouse-based PDX models. CAM PDX lines were established from primary patient-derived tumors; breast cancer PDX lines maintained in immunodeficient mice; and from cryopreserved tumor specimens.

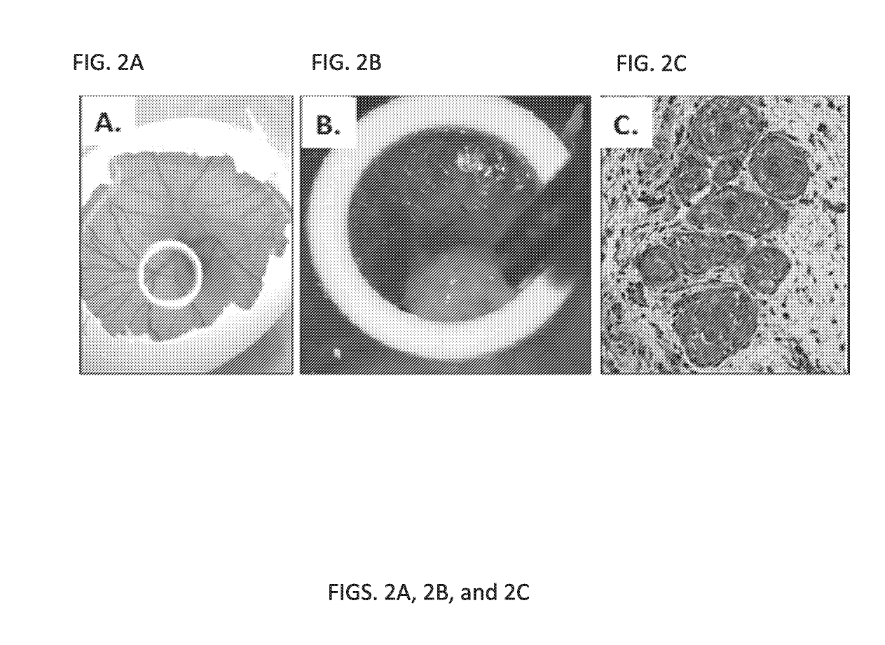

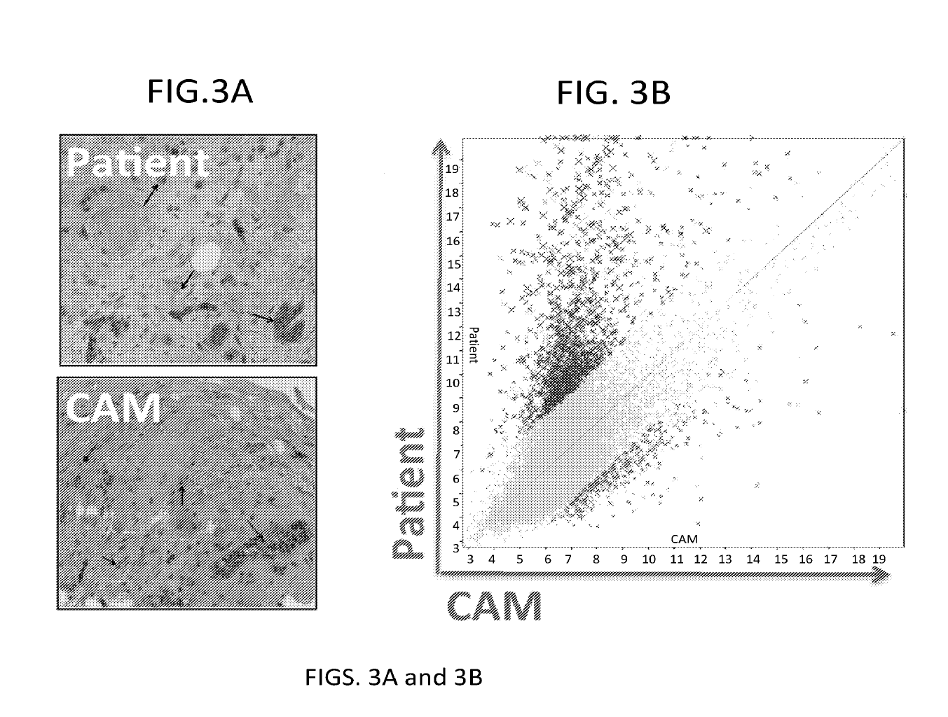

Successfully established lines (FIGS. 2, 7, and 8) demonstrate evidence of vascularization, growth (as ascertained by macroscopic and/or histologic assays, for example), and proliferation (as ascertained by Ki67 positivity, for example). Most importantly, the majority of initial (F0) grafts that “take” on CAM can be serially passaged across multiple recipient eggs (at least 4-7 passages for most of the lines tested) while maintaining morphologic stability and growth potential. There is also concordance of gene expression profiles between index tumor and CAM-PDX (FIG. 3). Establishing tumors on CAM is efficient and inexpensive compared to establishing mouse-based PDX, making it feasible to engraft multiple fragments from spatially distinct tumor regions. Therefore, the initial data indicates that CAM-based PDX is an ideal model system for capturing ITH by establishing and serially passaging multiple lines from different geographical tumor regions.

In certain embodiments, the chick chorioallantoic membrane (CAM) provides an ideal model system for development of a robust and scalable PDX approach capable of efficiently capturing intratumoral heterogeneity. In particular embodiments, in vivo growth of patient-derived tumors on CAM will recapitulate many aspects of the parent/primary tumor, including treatment sensitivity. CAM-based PDX may be utilized as a platform for comprehensive determination of tumor drug sensitivities and likelihood of therapeutic failure due to treatment resistance.

In particular embodiments, the capturing of intratumoral heterogeneity with CAM-based PDX is characterized by generating genetic and epigenetic tumor profiles from CAM-based PDX and comparing to parent (patient-derived) tissue. This may be accomplished by collecting tumor samples drawn from multiple spatially distinct intratumoral locations across a series of primary HNSCC tumors. A portion of each sample is used to determine baseline genomic/epigenomic profiles, and the remainder used to establish CAM-based PDX. For each dimension of assessment, the degree of initial intratumoral diversity is determined, and stability of these profiles is assessed across serial CAM passages. Thus, in specific embodiments there is capture of intratumoral heterogeneity with CAM-based PDX, and this diversity is preserved across multiple tumor passages.

One can perform whole-exome sequencing (WES) to determine the degree of baseline intratumoral heterogeneity and stability of genomic diversity on CAM-based PDX across multiple serial passages. One can subject patient-derived and CAM-based PDX tissue to epigenomic deconvolution (EDec). EDec provides a snapshot of epigenomic and transcriptional changes and also allows quantitative assessment of the relative contributions of cancer, stromal, and immune cells making up the tumor tissue.

Avatar-based precision medicine approaches rely on concordance of primary tumor and PDX sensitivity to candidate treatment approaches. One can determine the initial feasibility of drug sensitivity testing with CAM-based PDX. Breast cancer (or any cancer) mouse-based PDX lines with previously-characterized drug sensitivity profiles are transferred to the CAM, in specific aspects. Once established on CAM, the tumors are treated with a panel of chemotherapeutic and targeted therapy agents, and the relative sensitivities of tumors growing on CAM to therapeutic agents are compared to their previously-determined sensitivity profiles as mouse xenografts. One can demonstrate the ability of CAM-based PDX to serve as an accurate predictor of tumor sensitivity to therapeutic agents and lay the foundation for follow-up studies comparing in ovo drug response to patient clinical responses.
Characterization of the Capturing of Intratumoral Heterogeneity with CAM-Based PDX.

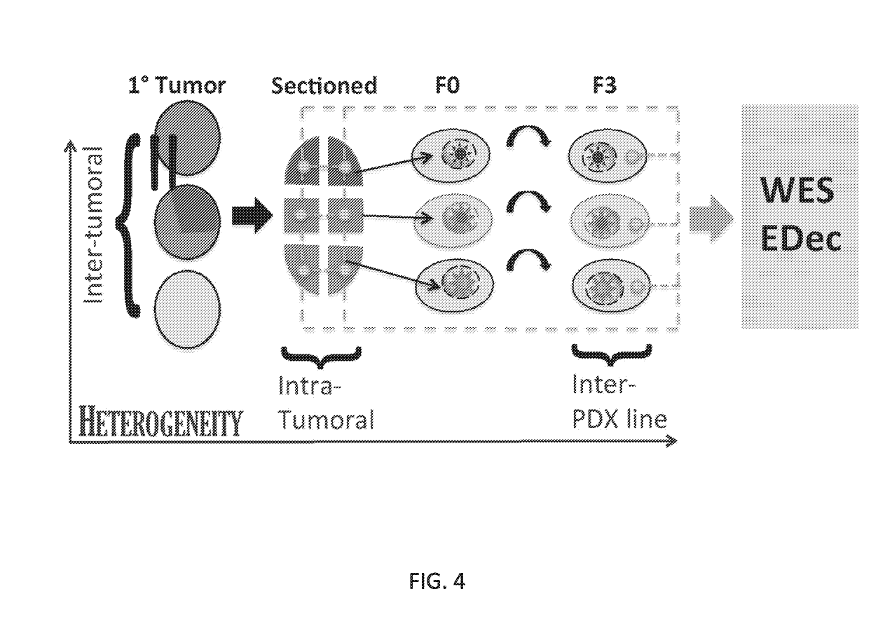

One can establish CAM-based PDX from multiple geographic regions across primary tumors collected from HNSCC patients and compare cellular, genomic, and epigenetic PDX profiles with those of primary tumor tissue (FIG. 4 shows an exemplary strategy).

Tumor collection, sectioning, and cryopreservation. HNSCC patients (although this would apply to any cancer) with primary tumors of adequate size and scheduled to undergo standard-of-care ablative surgery are used as an example of a source for the appropriate tissue collection/banking protocols. Tumor tissue in excess of that required for pathological analysis is harvested in the operating room and transported in chilled antibiotic-containing medium to the appropriate location. Tumors are sectioned along orthogonal axes to generate tissue samples from 3-8 geographically distinct tumor regions (depending on tumor size and viability). Half of each tissue sample is immediately frozen as baseline patient-derived tissue for future analyses, and the remaining tissue is engrafted on CAM on the day of harvest or cryopreserved in DMSO-containing medium for future grafting. This will permit generation of matched patient-derived and CAM-based tumor pairs for comparative genomic, proteomic, and epigenetic analysis.

Tumor engraftment onto CAM and serial passage. Patient- or PDX model derived-tumors are grafted on the chorioallantoic membrane (CAM) of embryonated eggs. In brief, tissue slivers are minced; the resultant slurry suspended in Matrigel® then grafted onto the CAM of fresh 6-7 day old fertilized chicken eggs. At 8-10 days post engraftment the tumors (F0 generation) are excised from the CAM, washed and prepared as described above for serial passages (F1-Fn). A portion of the tumor specimen from each passage may be cryopreserved for subsequent histological and molecular analyses to establish concordance with original tumors, for example.
Determination of the Degree of Baseline Intratumoral Heterogeneity and Stability of Genomic Diversity on CAM-Based PDX Across Multiple Serial Passages by Performing Whole Exome Sequencing.

Whole exome sequencing (WES) of primary and CAM-PDX tumor tissues. Genomic DNA is extracted from primary patient-derived tissue and F3 CAM serial passages and processed with, for example, TruSeq Exome Enrichment (FC-121-1048) and Nextera Exome Enrichment (FC-140-1003) kits to build the sequencing libraries. DNA is subjected to paired-end whole-exome sequencing using Illumina HiSeq2500 instruments. The sequence reads are processed and analyzed, for example using BaseSpace, an Illumina genomics computing environment for next-generation sequencing (NGS) data analysis and management.
Determination of the Feasibility of Drug Sensitivity Testing with CAM-Based PDX.

One can generate CAM-based PDX lines from established mouse-based breast PDX lines (see Table 1) with known drug sensitivities, and compare relative sensitivity of CAM- and mouse-based PDX lines to a panel of chemotherapeutic agents.

TABLE 1
Previously-characterized mouse-based breast cancer PDX lines and
drug sensitivity profiles.

| | | Treatment Response | | |
|---|---|---|---|---|
| PDX Line | Tumor Source | Docetaxel | Doxorubicin | Carboplatin |
| BCM-2147 | Pre-treat Bx | Resistant | Resistant | Sensitive |
| BCM-6257 | Pre-treat Bx | Sensitive | Resistant | Sensitive |
| BCM-4013 | Pre-treat Bx | Resistant | Resistant | Resistant |

CAM-PDX drug response assays. Mouse-based PDX lines are transferred from cryopreserved tissue to CAM and passaged for at least two additional generations (F3) before testing. The sensitivity of PDX lines grown on CAM to common breast cancer chemotherapeutic agents with differing mechanisms of action—docetaxel, doxorubicin, and carboplatin (for example)—are established. One can test a wide range of drug concentrations across five-fold dilutions to establish an appropriate treatment range; however, in certain embodiments concentrations similar to those effective against cells growing in 2D cell culture are also effective against cells growing on CAM. The study agent, or vehicle control, is incorporated into the initial tumor/Matrigel® slurry implanted onto CAM, and an additional 30 uL added every other day for the 10-day duration of the study (this volume and schedule was empirically determined in initial studies with tumor cell lines). On day 10 of growth on CAM, tumors are assessed for macroscopic size (white light photography and digital image analysis with ImageJ) and 3-D volumetric tumor measurement by MRI (FIG. 4). Tumors may be harvested and cryopreserved for later assessment of histological integrity, proliferation, and apoptosis.

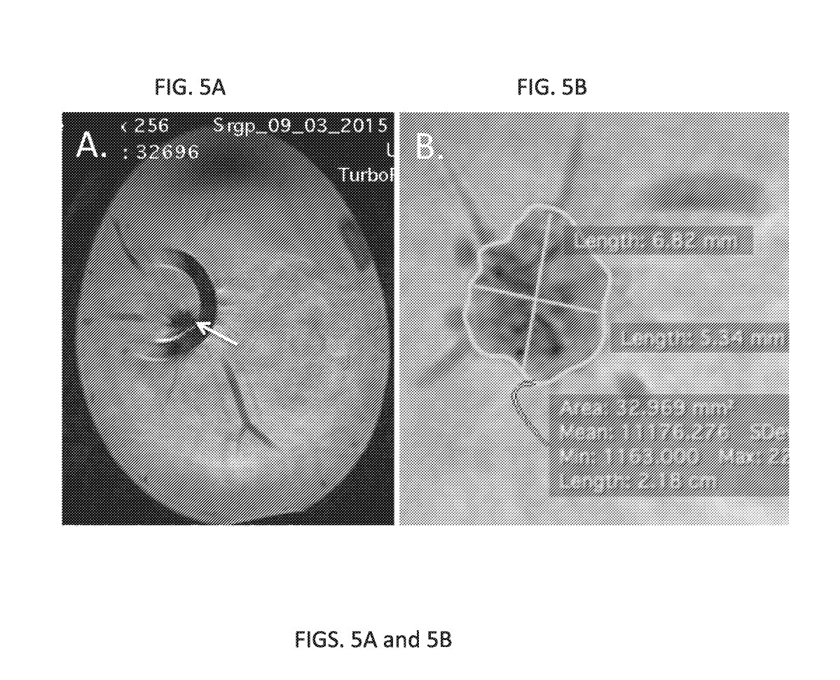

In ovo MRI analysis and volumetric assessment. MRI on eggs bearing viable CAM PDX tumors are performed. In specific aspects, the images are acquired with a 9.4T, Bruker Avance I Biospec Spectrometer, 21 cm bore horizontal scanner with a 72 mm volume resonator (Bruker Biospin, Billerica, Mass.), and acquired with a 3D Turbo-RARE rapid-acquisition sequence with an isotropic spatial resolution of 117 microns (FIG. 5). Volumetric assessments are performed with AMIRA image processing software packages to determine tumor areas and volumes from the 3D MRI datasets, in specific aspects.
Statistical Approach.

Volumes of triplicate in ovo tumors may be averaged to determine the central tendency and range of variation for each condition, and between-group differences determined by student's T test, using p<0.05 as the threshold for significance. For each PDX line in ovo dose response curves for each drug are fit, and significance of differences are determined by nonlinear regression. Rank-order of drug sensitivities in ovo is established and compared to that previously established in mouse-based PDX.

### Example 4

### Ovotars—an Efficient Approach to Patient-Derived Xenografts

Precision or “personalized” cancer medicine focuses on using individual tumor and/or host characteristics to select optimal patient-specific therapeutic regiments. Patient derived tumor xenografts (PDX), typically grown in immunodeficient recipient mice, provide a versatile and renewable source of PDX tumor tissue and a platform for testing tumor sensitivity to different therapies in vivo. However, the development of additional xenograft platforms based on phylogenetically simpler host organisms could supplement mouse-based PDX by enhancing the efficiency and scalability of PDX approaches.

Materials and Methods

Tumor specimens derived from patient- and mouse PDX-derived tumors were obtained under IRB and IACUC protocols, morcelized, and placed in an optimized suspension of PBS and Matrigel®, prior to explanting onto the vascularized CAM of 6-7 day chick embryos. Explants were incubated at 37° C. with 60-70% humidity. At day 17, chicks were humanely euthanized and tumors processed for downstream applications

Results

The results successfully demonstrate the efficiency and reproducibility of the CAM-PDX model across multiple tumor types. There was successful establishment of 50 CAM PDX lines derived from 10 patients with 5 different tumor types, including head and neck squamous cell carcinoma (HNSCC), breast cancer, adnexal carcinoma, papillary thyroid cancer and skin squamous cell carcinoma. The overall take rate of 80% compares favorably to that in mouse-based PDX models.

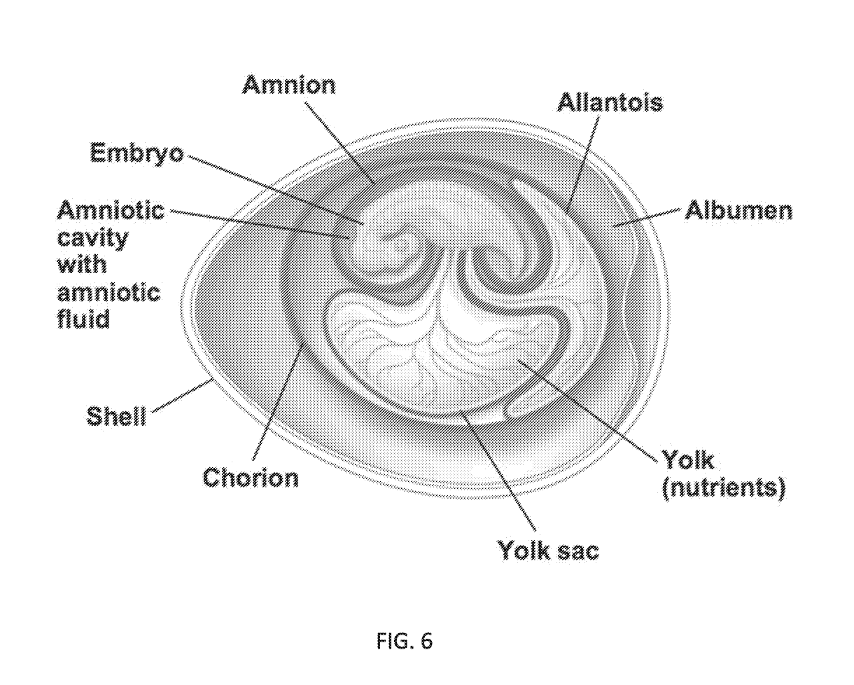

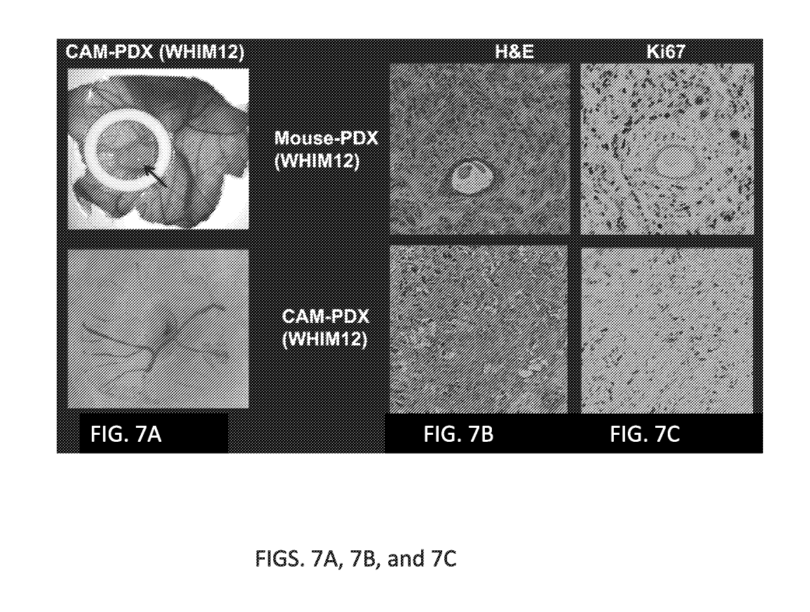

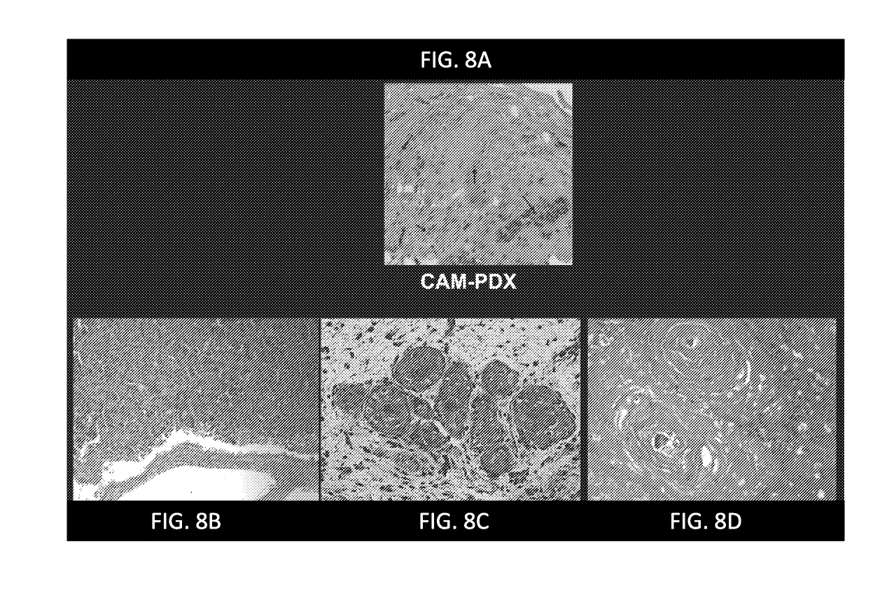

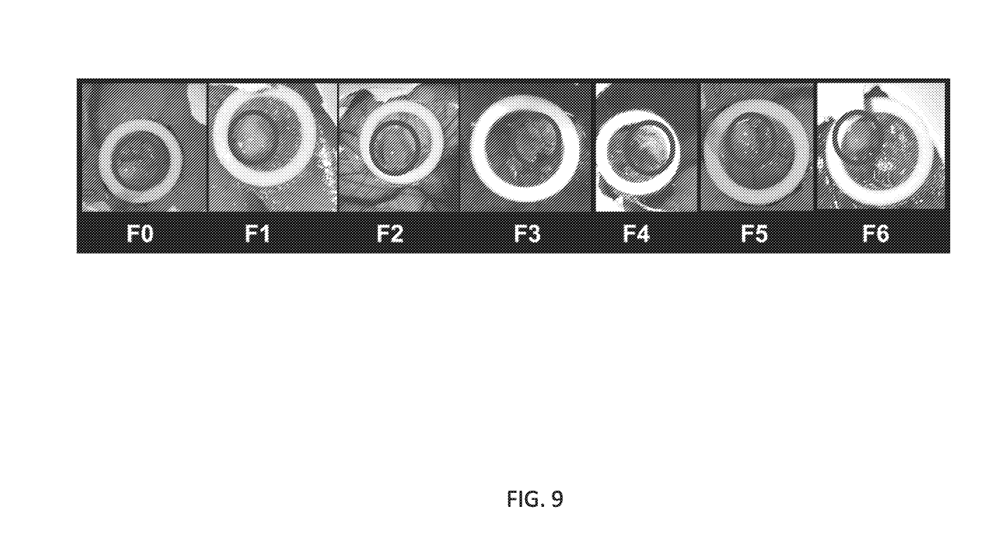

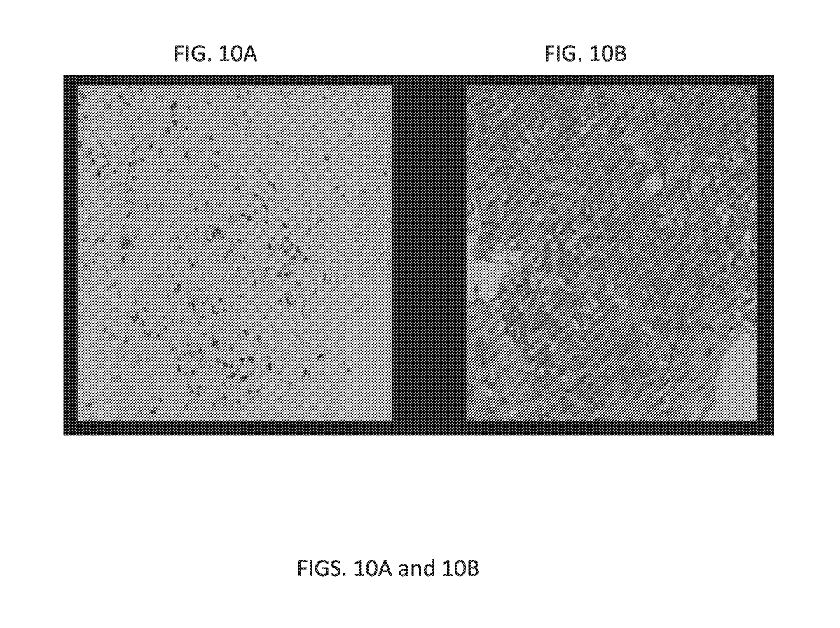

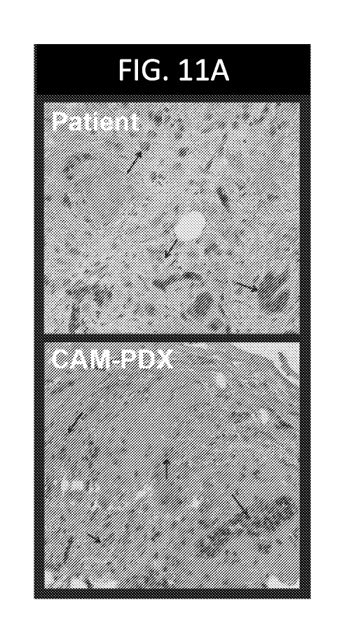

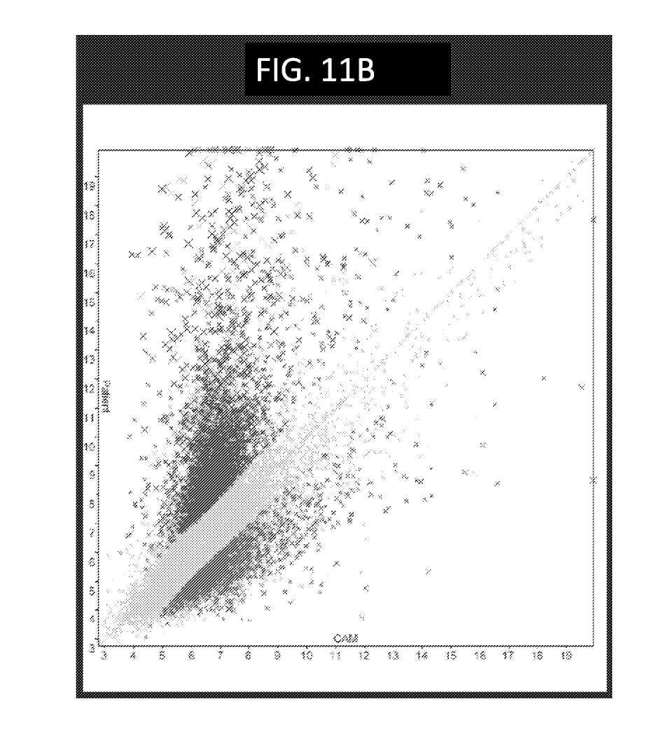

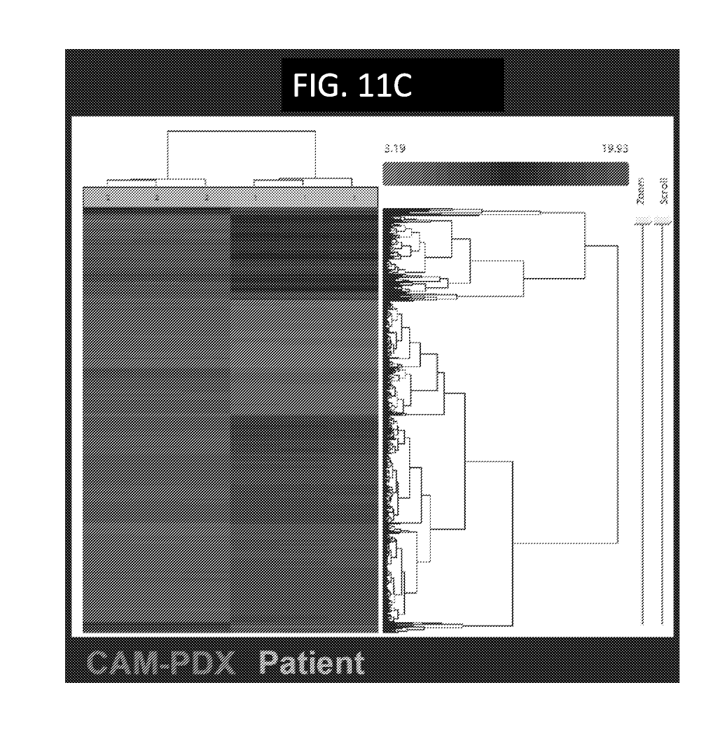

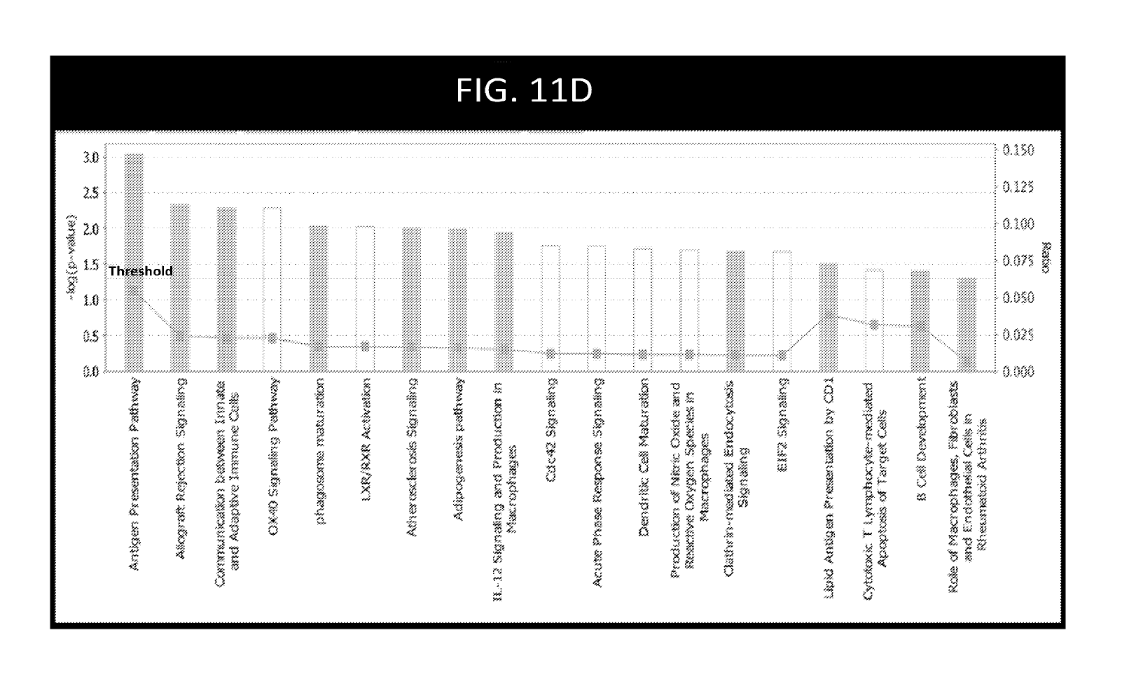

FIG. 6 shows the CAM surrounding a young check embryo. FIG. 2 provides an example of a CAM xenograft model. FIG. 7 shows microscopic images of CAM-based breast PDX. FIG. 8 demonstrates pan-cancer xenografts derived from patient tumors (patient to egg, or Patient-Egg). FIG. 9 provides images of serial passage and FIG. 10 shows revival of cryopreserved CAM-PDX. FIG. 11 shows stability of gene expression profile between index tumor and CAM-PDX. FIG. 5 demonstrates in ovo MRI analysis and volumetric assessment.

### Example 5

### Examples of CAM System Configurations

In one embodiment, there is an “established” horizontal method as follows (although uses of the horizontal method as described herein were not established):

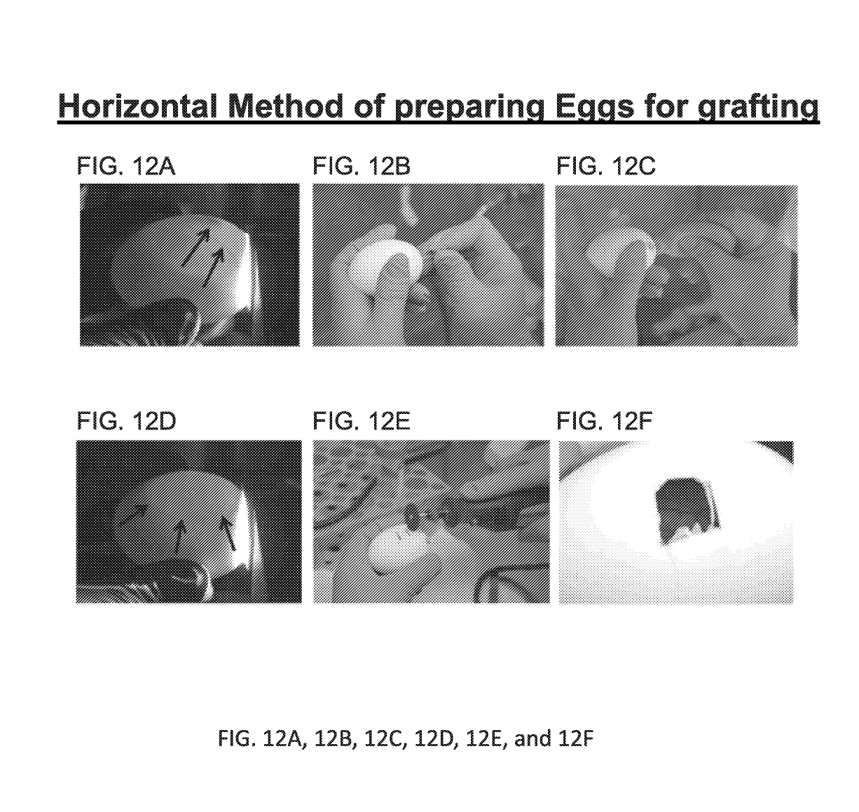

At day 6-8 post-fertilization, embryonated sterile eggs incubated at 37° C. with 80% humidity are transferred to an egg tray with the air-sac end upwards and placed in a laminar flow hood. An area between two blood vessels is identified and labeled. The eggs may be positioned horizontally with the air sac end upwards. A micro vent (3 mm deep) is made on the air sac end of the egg shell, and a second vent is made at the labeled mark with a sterile pushpin. A safety bulb is pressed against the hole over the label mark due to the suctioning, the CAM is “dropped” and separated from the eggshell. Next the egg is held in one hand and using a Dremel rotary tool with a 15/16 inch wheel attachment,two transverse cuts (2 cm×1 cm) are made on the shell on the region above the CAM without touching the CAM, and the cut shell is removed gently with a sterile forceps. The shell window is sealed using a sticky tape folded over itself at one end (FIG. 12). The egg is placed on an egg tray and the tray returned to the incubator without rotation for 2-3 hours before inoculation.

In one embodiment of the disclosure, a “vertical” method of the CAM model is utilized, which may be generated as follows:

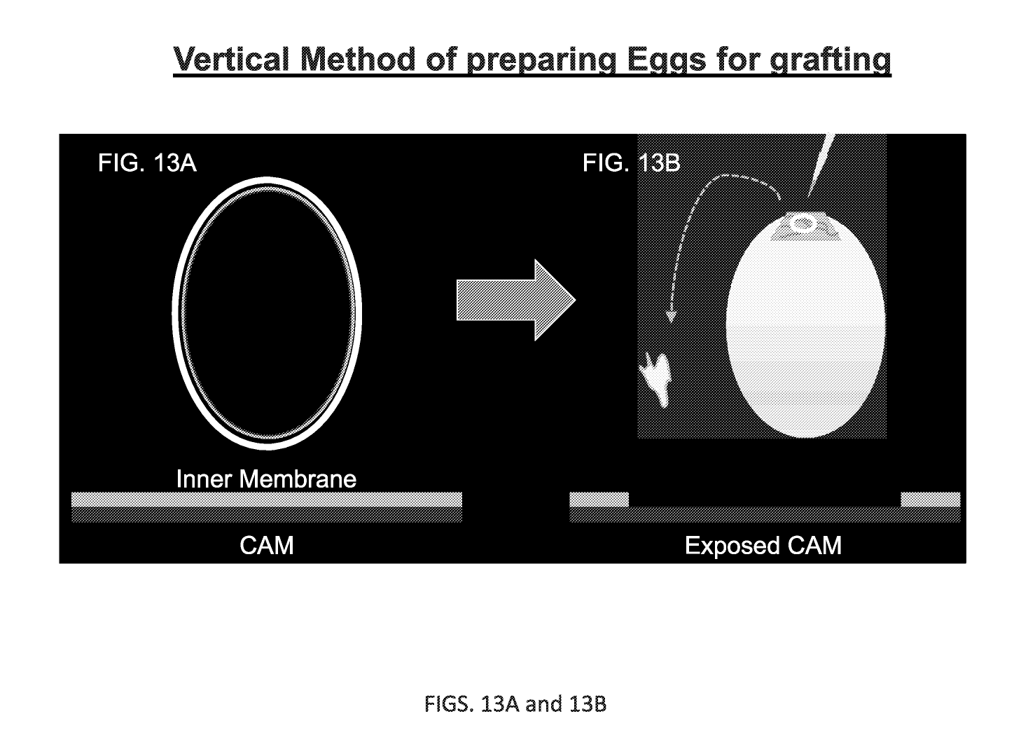

At day 6-8 post-fertilization, embryonated sterile eggs incubated at 37° C. with 80% humidity are transferred to an egg tray with the air-sac end upwards and placed in a laminar flow hood. Using a Dremel rotary tool with a 15/16 inch wheel attachment, two transverse cuts (2 cm×1 cm) are made on the shell at the air sac end. The cut egg shell is removed to reveal the inner membrane that is closely associated with the CAM. Using a bent forcep the inner membrane layer is carefully peeled away from the top of the CAM to reveal the vasculature beneath. It is critical to avoid puncturing or agitating the CAM (this leads to bleeding, and eventual death of the embryo). The shell window is sealed using a sticky tape folded over itself at one end (FIG. 13). The egg is placed on an egg tray and the tray returned to the incubator without rotation for 2-3 hours before inoculation.

### Example 6

### Various CAM System Applications

### Pan-Cancer Xenografts (Patient Biopsies/Resections)

In certain embodiments, CAM systems are employed for culturing tissue, such as cancer tissue from an individual. The source of the tissue may come directly from an individual that has cancer, or it may come from another tissue culture system. The tissue culture din the CAM system may be from the same or diverse tumor types. One source of the tissue includes patient biopsy or resection. The resection may be the result of surgery that removes part or all of a tumor, and in some cases the CAM system is utilized for analysis of the tumor tissue to determine a suitable treatment(s) for the individual from which the tumor tissue was obtained. The tissue placed in the CAM system may come directly from the individual, or it may have first been processed in another tissue model (including a model wherein the model is of another species).

The tissue from the individual may be first manipulated prior to inoculation of the CAM model. To prepare for inoculation, a vial of grafting solution* (Table 2) is placed on ice to prevent polymerization.

TABLE 2
Composition of grafting material (matrigel)

| | Range of GF | | Routinely used | |
|---|---|---|---|---|
| Growth Factor (GF) | Concentration | | Concentration | |
| EGF | 0.5-1.3 | ng/mL | <0.5 | ng/mL |
| bFGF | <0.1-0.2 | pg/mL | <0.1-0.2 | pg/mL |
| NGF | <0.2 | ng/mL | <0.2 | ng/ml |
| PDGF | 5-48 | pg/mL | <5 | pg/mL |
| IGF-1 | 11-24 | ng/mL | 5 | ng/mL |
| TGF-B | 1.7-4.7 | ng/mL | 1.7 | ng/mL |

The material is approximately 60% laminin, 30% collagen IV, and 8% entactin. It also contains heparan sulfate proteoglycan (perlecan), TGF-β, epidermal growth factor, insulin-like growth factor, fibroblast growth factor, tissue plasminogen activator, and other growth factors which occur naturally in the Engelbreth-Holm-Swarm (EHS) murine sarcoma (as an example of a tumor). There is also some residual matrix metalloproteinases.

Tumor specimens derived from patient- and mouse PDX-derived tumors are cut into 100-200-mg pieces (for example), and incubated in minimal essential medium (MEM) supplemented with antibiotics for 15-30 minutes. The morcelized tumor pieces are placed in a suspension of PBS (containing calcium and magnesium) and grafting solution. The ratio of PBS to grafting solution is optimized at 1:1. The morcelized mix is then explanted onto the vascularized CAM of 6-8 day chick embryos. Explants were incubated at 37° C. with 60-70% humidity. At day 17, chicks are humanely euthanized and tumors processed for downstream applications. The inventors have successfully generated xenografts from diverse cancer types including breast, head and neck (laryngeal, oral squamous, tonsil), adnexal, skin and cervical cancers (Table 3). Tumor source material includes patient and mouse PDX-derived tumors, in addition to different cancer cell lines. Note: An optimized grafting solution can be supplemented with tumor/cancer specific growth factors, and essential nutrients (e.g., estradiol in breast cancer)

TABLE 3
Pan Cancer PDX derived using the technology

| Tumor Type | Take Rate/Patient | Viable Models | Take Rate |
|---|---|---|---|
| HNSCC | 12/13 | 63 | 92% |
| Breast | 6/6 | 11 | 100% |
| Thyroid | 1/2 | 2 | 50% |
| Cervical | 1/2 | 2 | 50% |
| Salivary | 2/2 | 12 | 100% |
| Skin/Adnexal | 2/2 | 16 | 100% |
| Renal Cell Carcinoma | 4/5 | 16 | 80% |
| TOTAL (PDX) | 28/32 | 122 | 87% |

### Serial Passage

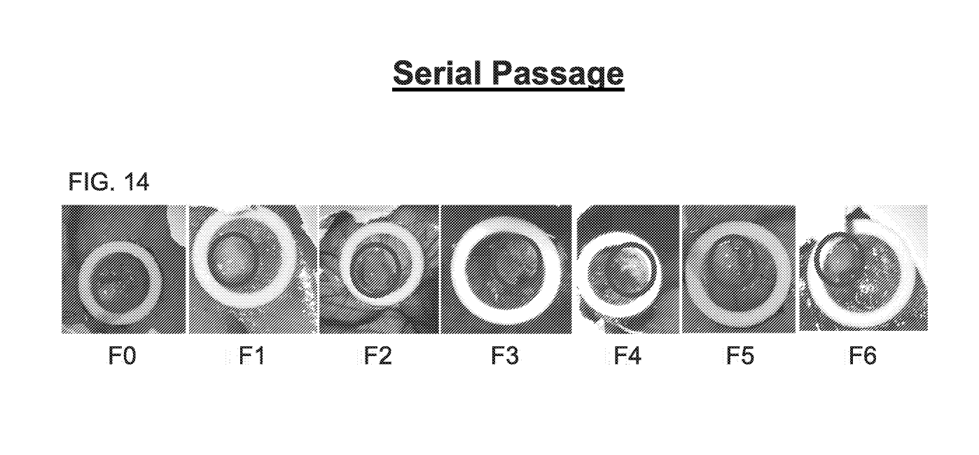

In some embodiments, the CAM models are utilized for serial passaging. For example, one can obtain tissue from an individual directly or it may be obtained from another model (including a model wherein the model is of another species). The ability to serially passage tumors derived from patient and PDX models (breast, oral squamous carcinomas, as examples) has successfully been demonstrated (FIG. 14). For this, one can use either of the two previously described methods of preparing the eggs (FIGS. 12 and 13), as examples.

In specific embodiments, the passaging is to a different egg. In at least some cases, part of the tumor is passaged, such as within a range of 25-50 mg of tumor tissue for passaging (although the tumor tissue may or may not be weighed prior to transfer). One may harvest the tumors and separate any egg membrane attached to it. The tumors are transferred to a suitable media, such as DMEM media containing 10% FBS. The tissue may be rinsed in a suitable solution, such as PBS with calcium and magnesium, for example, prior to grafting on the egg.

### Shuttle PDX Model (Patient-Egg-Mouse)

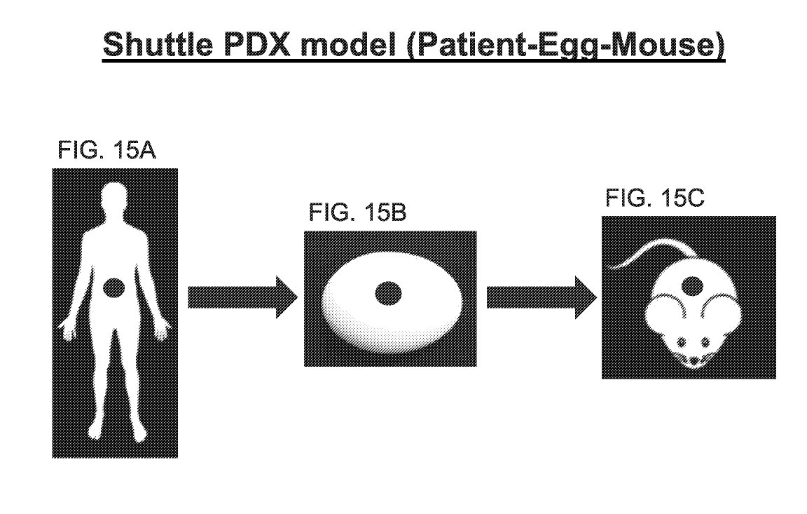

The CAM model serves as a viable host for generating xenografts for tumors obtained from the mouse PDX models (PDX-Chicken), and directly from patients (Patient-Chicken). One can also generate mouse PDX from chicken-derived tumors, which are of human origin (Patient-Chicken-Mouse PDX) (FIG. 15).

In some cases tissue from a PDX-Chicken model is transferred to a mouse model because mouse models are not always successful in successfully grafting human derived tumors. This might happen because of a number of factors that include limited tumor sample size. Growing them on the eggs will enable expansion of the tumor material and condition the tumor to grow successfully outside of the human host, which would facilitate its graft onto the mouse model.

### PDX Sensitivity Testing Platform

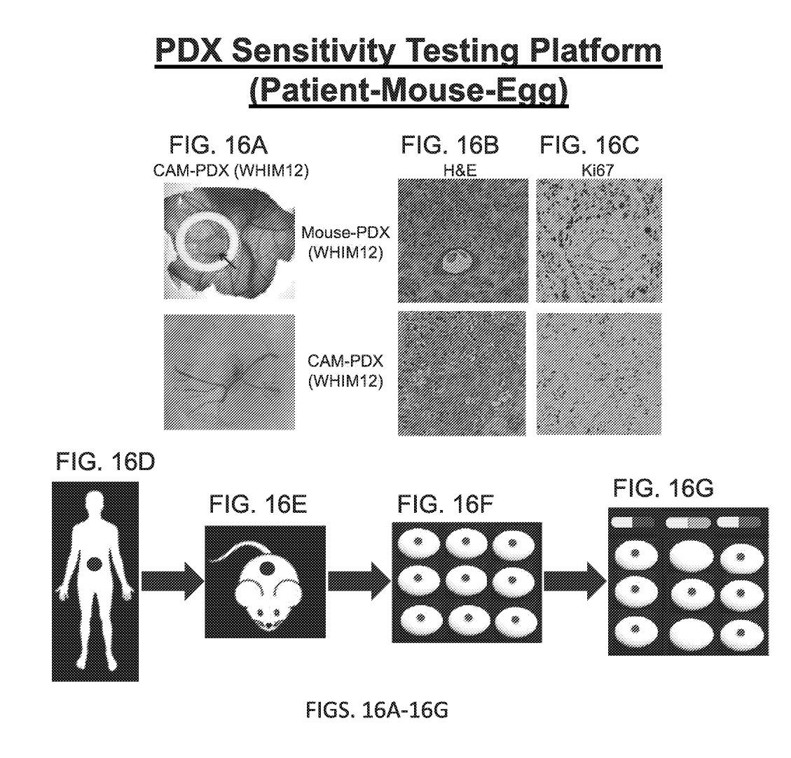

In some embodiments, one can grow mouse-derived tumors on the egg to conduct drug sensitivity and other tests, such as biomarker development, co-clinical trials, and personalized medicine tests. in a rapid and efficient manner. The egg model allows tumors to grow faster than the mouse, for example 1 week compared to months. The workflow includes growing primary tumors in mouse models, harvesting them and growing tumors on multiple eggs; followed by drug treatments to rapidly screen for drug response (FIG. 16). For example, a drug to be tested (a compound with unknown or uncharacterized or poorly characterized capabilities for use as a therapeutic drug) may be provided in sufficient quantity to the tumor growing on the egg to determine if the tumor is thereafter reduced in size, although in some cases a range of doses of a known drug or drug to be tested are employed to determine a suitable therapeutic dosage. In some cases, a combination of drugs and/or test drug candidates are provided to the tumor to determine if there is a combinatorial effect, synergistic effect, no effect, or a deleterious effect, for example.

Other than testing tumor size with a model of the disclosure, one can test an increase in apoptotic markers and/or decrease in proliferative markers, in addition to monitoring alterations in tumor specific markers for a given type of cancer, for example. One may also assess tumor invasiveness, metastasis, and/or angiogenic potential.

### Deriving Primary Cell Lines

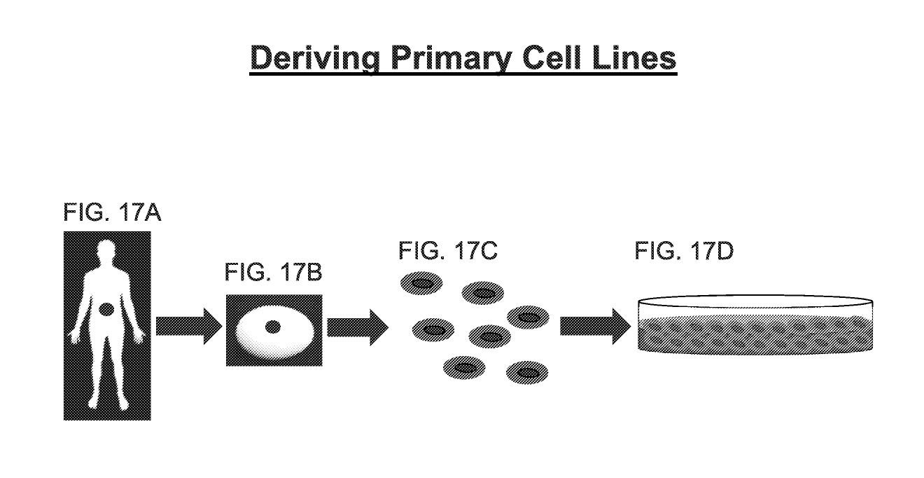

Although in some cases cell lines are utilized as the source of the tumor, in other cases an established tumor on the CAM model is the source of cells to establish a cell line. Patient derived tumors have been successfully grafted on the CAM, and the xenografts have been used to derive primary cell lines (FIG. 17). The tumor cells derived from the xenografts have been used for multiple applications, including flow cytometry and viral transduction. In cases where tumor material is limiting, the CAM-based method can generate additional tumor material from the source, thereby facilitating downstream applications. In some cases, the tumor material is analyzed both before and after generation of the cell lines.

### Multiplexing: One Egg—Multiple Tumors

The inventors have demonstrated the capability to grow multiple tumors on a single egg by using custom designed “tumor casts”. The casts are made of biologically inert material that demarcates the area within which the tumor grows. Using these, the inventors have successfully grown 4 tumors on a single egg, and in specific embodiments more than 4 tumors, such as at least 6 tumors, on a single egg may be grown. In at least some cases a limitation to the number of tumors per egg is determined by the size of the window that is cut in the shell. Additionally, we run the risk of severing some of the vasculature on the sides if we try to have wider windows. Of course, the number of tumors per egg is determined at least in part by the appropriate size of casts to separate them.

### 2D to 3D Conversion of Cancer Cell Lines (Pan Cancer)

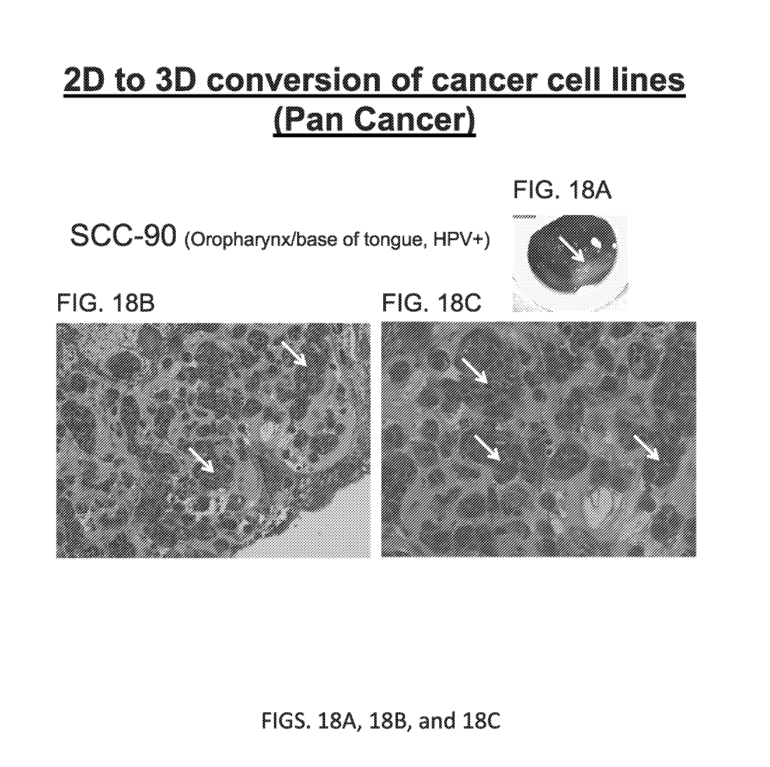

Although in some cases an established tumor in a CAM model is the source of cell lines, in other cases cell lines are utilized as the source of the tumor for the egg. The inventors have successfully demonstrated the capability to convert immortalized cancer cell lines from their native 2D configuration in liquid culture to highly vascularized 3D tumors using the present novel method of preparing the CAM (although the cell lines do not need to be immortalized). Although in some embodiments, an optimized ratio of grafting solution to cancer cells is utilized (for example, 1:1, 1:1.5) (FIG. 18), in other cases other ratios are employed (such as 1:1.1, 1:1.2, 1.25, 1:1.3, 1:1.4, 1:1.6, 1:1.7, 1:1.75, 1:1.8, 1:1.9, 1:2, and so forth.

### Intermediate-High Throughout Platform for 2D to 3D Conversion of Cancer Cell Lines and Xenograft Cultures

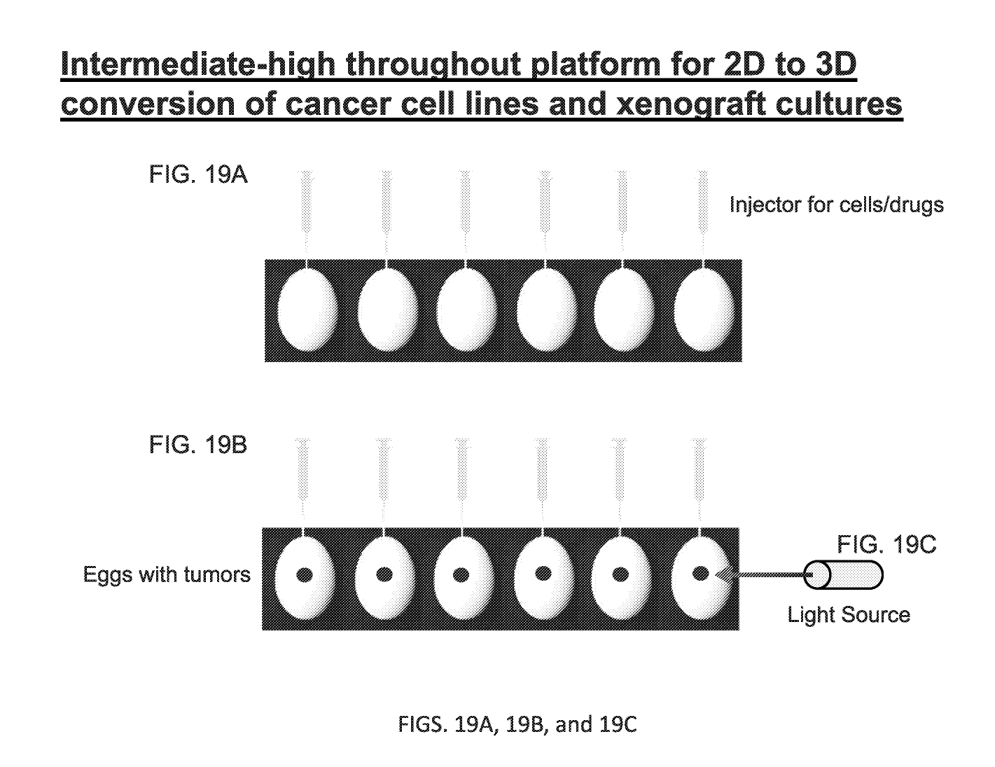

The novel method of culturing tumor xenografts and converting 2D cells to 3D tumors can be scaled up by automation (FIG. 19), in some embodiments. This would comprise of a mechanism that would a) cut a window on vertical eggs; b) separate the inner membrane from the CAM; c) deliver cell/tumor suspension onto the CAM; d) deliver drugs/small molecules to the 3D tumors; and e) image the tumors and quantitate growth parameters. In some cases, the automation component would transfer tumor(s) to another site on the same egg or transfer them to another egg.

Thus, in specific embodiments one can harvest and process tumors for xenografting them onto different eggs. A fluorescent detection system to ascertain which areas are most viable for grafting may be utilized as a companion technology, for example.

Vertically-placed eggs or horizontally-placed eggs may be utilized in automation designs.

### Combinatorial Adenovirus and CAR T-Cell Therapy in 3D Tumors on Eggs

In some embodiments, the CAM-based model is utilized to test the effectiveness of one or more cancer therapies, which may be of any kind, including small molecules, proteins (including antibodies), nucleic acids, oncolytic viruses, and therapeutic cells, including therapeutic immune cells. Therapeutic cells may be of any kind, including modified T cells that may or may not have a targeting moiety to target them to a particular tumor antigen, for example. A specific example of therapeutic T cells includes T cells that are modified to express a chimeric antigen receptor (CAR), usually having fusions of single-chain variable fragments (scFv) derived from monoclonal antibodies, fused to CD3-zeta transmembrane and one or more endodomains, such as one or more costimulatory endodomains.

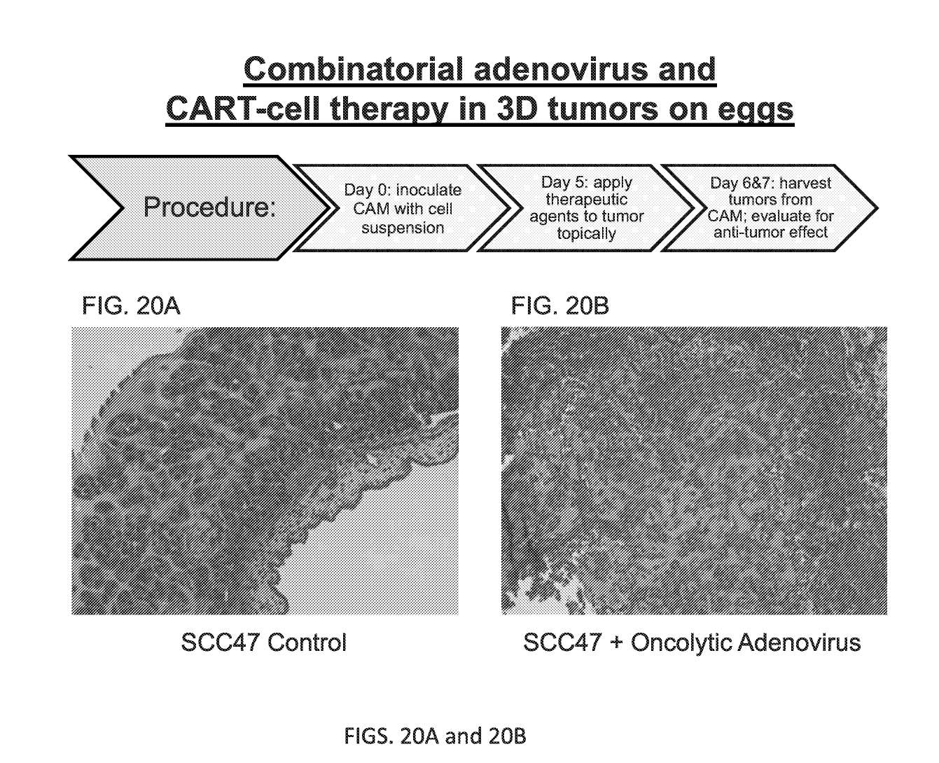

As an example, the inventors have successfully used oncolytic adenoviruses to target 3D tumors (converted from 2D immortalized cancer lines) (FIG. 20). Initial studies indicate that the oncolytic adenoviruses are effective against the 3D tumors. These findings indicate that one can use these, including in combination with immune therapy of any kind (e.g., CART cells) to increase the targeting and therapeutic efficiency. When testing two more potential drugs, they may be delivered to the model in the same or different administrations.

### Companion Assay: MRI Based Imaging Method for Tumor Assessment

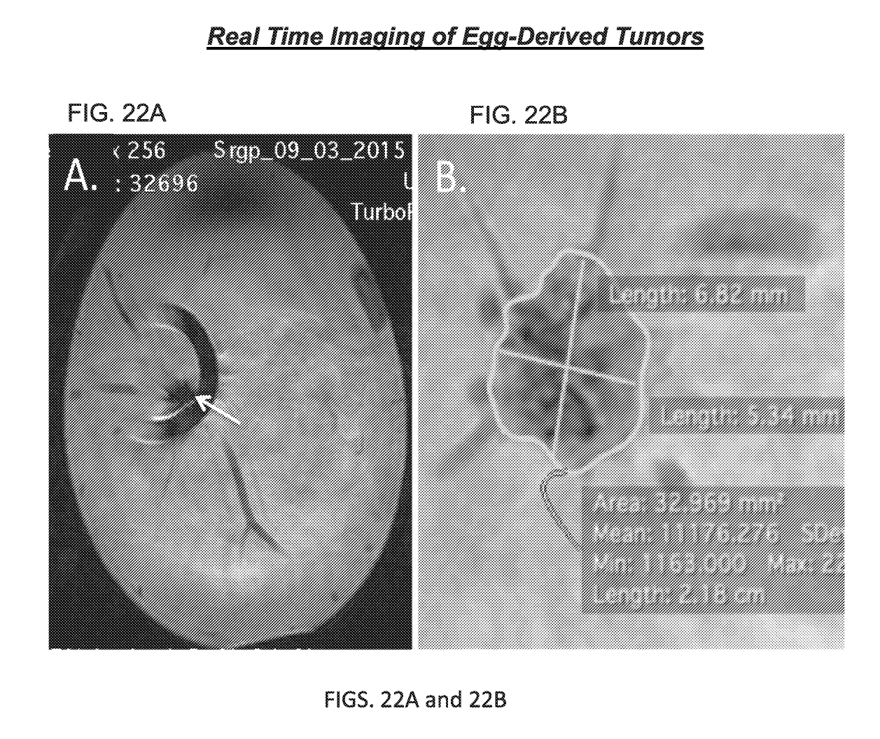

In some cases, following establishment of the tumor on the CAM-based model, one or more characteristics of the tumor is analyzed, such as size, viability, vasculature, angiogenesis, or a combination thereof. In specific embodiments, the CAM based model allows measurement of tumor volumes and other parameters using an MRI-based imaging method that can perform real time imaging of tumors in a short time (FIG. 22). In other cases one may utilized IVIS, CT/PET, and/or fluorescent imaging platforms in addition to or in lieu of MRI. FIG. 22 shows a representative in ovo MRI image of breast cancer PDX, demonstrating a teflon ring, beginning of tumor nodule (arrow), and feeding vessels. The figure shows a close up of a 0.5 mm slice with tumor ROI identified for quantitative analysis. Peripheral feeding vessels are also visible.

### Companion Assay: Genomic/Proteomic/Metabolic Profiling

One embodiment allows for analysis of genomic, proteomic, or metabolic signatures of an established tumor on a CAM-based model. The CAM-based model may or may not comprise a xenograft-derived tumor.

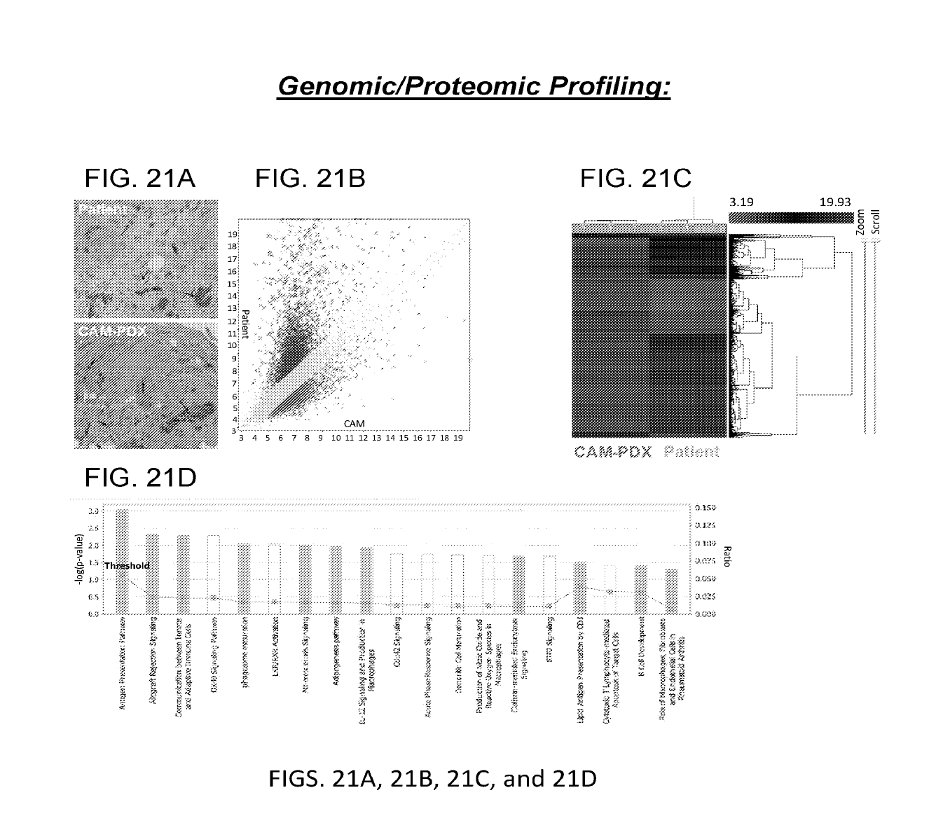

The inventors recently generated a xenograft model for a patient derived tumor (breast cancer, as an example), and obtained its detailed genomic profile using Affymetrix Gene Chip arrays. The results indicate that there is a very high degree of correlation between the patient and the CAM derived tumors (FIG. 21), thereby establishing validity for drug sensitivity and efficacy assays. The entire process was completed within 2-3 weeks, as compared to the existing mouse models that can take months. Similar data was generated using mass spectrometry-based proteomics to identify the percentage of human proteins in the egg-derived tumors.

### Companion Technology: Cryopreservation and Revival of Frozen Tumor Material:

In some cases, tumor material from an established CAM-based model is preserved for future use, either by the individual(s) that generated the model and/or by others. In at least specific methods, the tumor tissue is preserved using suitable temperature.

In certain cases, the tumor material on the CAM is appropriately processed prior to preservation. For example, tumors grown on CAM are carefully separated from the underlying CAM membrane and matrigel. Tumors are washed in PBS (—calcium and magnesium), and incubated immediately into DMEM media containing 10% FBS for 5-10 minutes. The tumors are subsequently transferred into a cryovial containing a freezing mixture of DMEM media with 10% FBS and 10% DMSO. The cryovials are frozen in a step-wise manner at −80° C. for 24-48 hours, and shifted to liquid nitrogen storage tanks for long time storage. Using this embodiment of optimized methodology, the inventors have successfully revived frozen tumor material from different cancer types stored at −80° C. Multiple xenografts have been established with consistently high take rates (70-80%). Frozen material includes freshly frozen tumors derived from patient, mouse and egg derived PDX models.

### Re-Derivation Protocol for Viably Frozen PDX Tissue:

The CAM-based models may be employed using frozen tissue as a source tissue, including frozen tissue that came from a CAM-based model (at one point in time) or from frozen tissue that was frozen directly from an individual, for example.

The cryovials are retrieved from liquid nitrogen and thawed immediately on ice. The freezing media is removed from the tube and 1 mL high glucose DMEM is added to tube. The tumor material is suspended in the fresh media and mixed well, before being dumped into a 15 ml conical tube containing 14 ml of high glucose DMEM. The tumor is washed thoroughly in the tube by repeated pipetting, and the media is discarded. The process is repeated 1-2 times with 15 mL of high glucose DMEM. After the final another 15 mL of high glucose DMEM is added and the tumor is placed on ice if transplanting immediately. Before grafting onto the CAM the tumor is washed thoroughly (2-3 times) in PBS containing calcium and magnesium to remove all traces of media.

### REFERENCES

All patents and publications mentioned in this specification are indicative of the level of those skilled in the art to which the invention pertains. All patents and publications herein are incorporated by reference to the same extent as if each individual publication was specifically and individually indicated to be incorporated by reference in their entirety.

Amin, Viren, R. Alan Harris, Vitor Onuchic, Andrew R. Jackson, Tim Charnecki, Sameer Paithankar, Sai Lakshmi Subramanian, Kevin Riehle, Cristian Coarfa, and Aleksandar Milosavljevic. 2015. “Epigenomic Footprints across 111 Reference Epigenomes Reveal Tissue-Specific Epigenetic Regulation of lincRNAs.” Nature Communications 6: 6370. doi:10.1038/ncomms7370.

Chalise, Prabhakar, Devin C. Koestler, Milan Bimali, Qing Yu, and Brooke L. Fridley. 2014. “Integrative Clustering Methods for High-Dimensional Molecular Data.” Translational Cancer Research 3 (3): 202-16. doi:10.3978/j.issn.2218-676X.2014.06.03.

Deryugina, Elena I., and James P. Quigley. 2008. “Chick Embryo Chorioallantoic Membrane Model Systems to Study and Visualize Human Tumor Cell Metastasis.” Histochemistry and Cell Biology 130 (6): 1119-30. doi:10.1007/s00418-008-0536-2.

Lopez-Rivera, Esther, Padmini Jayaraman, Falguni Parikh, Michael A. Davies, Suhendan Ekmekcioglu, Sudeh Izadmehr, Denái R. Milton, et al. 2014. “Inducible Nitric Oxide Synthase Drives mTOR Pathway Activation and Proliferation of Human Melanoma by Reversible Nitrosylation of TSC2.” Cancer Research 74 (4): 1067-78. doi:10.1158/0008-5472.CAN-13-0588.

Malaney, Prerna, Santo V. Nicosia, and Vrushank Davé. 2014. “One Mouse, One Patient Paradigm: New Avatars of Personalized Cancer Therapy.” Cancer Letters 344 (1): 1-12. doi:10.1016/j.canlet.2013.10.010.

Murphy, J. B. 1913. “TRANSPLANTABILITY OF TISSUES TO THE EMBRYO OF FOREIGN SPECIES: ITS BEARING ON QUESTIONS OF TISSUE SPECIFICITY AND TUMOR IMMUNITY.” The Journal of Experimental Medicine 17 (4): 482-93.

Petruzzelli, G. J., C. H. Snyderman, J. T. Johnson, and E. N. Myers. 1993. “Angiogenesis Induced by Head and Neck Squamous Cell Carcinoma Xenografts in the Chick Embryo Chorioallantoic Membrane Model.” The Annals of Otology, Rhinology, and Laryngology 102 (3 Pt 1): 215-21.

Pribluda, Ariel, Cecile C. de la Cruz, and Erica L. Jackson. 2015. “Intratumoral Heterogeneity: From Diversity Comes Resistance.” Clinical Cancer Research: An Official Journal of the American Association for Cancer Research 21 (13): 2916-23. doi:10.1158/1078-0432.CCR-14-1213.

Ribatti, Domenico. 2014. “The Chick Embryo Chorioallantoic Membrane as a Model for Tumor Biology.” Experimental Cell Research 328 (2): 314-24. doi:10.1016/j.yexcr.2014.06.010.

Roadmap Epigenomics Consortium, Anshul Kundaje, Wouter Meuleman, Jason Ernst, Misha Bilenky, Angela Yen, Alireza Heravi-Moussavi, et al. 2015. “Integrative Analysis of 111 Reference Human Epigenomes.” Nature 518 (7539): 317-30. doi:10.1038/nature14248.

Somasundaram, Rajasekharan, Jessie Villanueva, and Meenhard Herlyn. 2012. “Intratumoral Heterogeneity as a Therapy Resistance Mechanism: Role of Melanoma Subpopulations.” Advances in Pharmacology (San Diego, Calif.) 65: 335-59. doi:10.1016/B978-0-12-397927-8.00011-7.

Zhang, Xiaomei, and Michael T. Lewis. 2013. “Establishment of Patient-Derived Xenograft (PDX) Models of Human Breast Cancer.” Current Protocols in Mouse Biology 3 (1): 21-29. doi:10.1002/9780470942390.mo120140.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of establishing tumor tissue in a model, comprising the steps of:
a) providing, obtaining, or producing one or more avian or reptilian chorioallantoic membrane egg models;
b) providing, obtaining, or producing one or more of the following:
1) cells from cancer cell lines;
2) tissue from one or multiple regions of a single tumor in a mammal;
3) tissue from one or multiple regions of one or more tumors in a mammal; and/or
4) tissue from a patient-derived xenograft model and/or a choriollantoic membrane egg model;
and
c) culturing the cells and/or tissue from b) on one or multiple locations of a single avian or reptilian chorioallantoic membrane egg model or on one or multiple locations of multiple avian or reptilian chorioallantoic membrane egg models.

2. The method of claim 1, further comprising the step of:
d) assaying the cultured tumor tissue.

3. The method of claim 2, wherein the cultured tumor tissue is passaged to another model one or more times.

4. The method of claim 3, wherein the other model is a chick chorioallantoic membrane (CAM) model, a mouse model, a frog model, a dog model, guinea pig model, hamster model, rabbit model, cat model, livestock model, fish model, or a rat model.

5. (canceled)

6. (canceled)

7. (canceled)

8. The method of claim 2, wherein the assaying comprises exposure of the cultured tumor tissue to a cancer therapy to be tested.

9. (canceled)

10. (canceled)

11. (canceled)

12. (canceled)

13. (canceled)

14. The method of claim 8, wherein the tumor tissue is obtained from the mammal prior to exposure to a cancer therapy for the individual, following exposure to a cancer therapy for the individual, or both.

15. The method of claim 8, wherein an effective amount of the cancer therapy for the tumor tissue is determined.

16. The method of claim 2, wherein the assaying comprises genomic profiling, metabolic profiling, and/or proteomic profiling.

17. The method of claim 2, wherein as a result of the method, the mammal from which the tumor tissue was originally derived is provided a suitable cancer therapy.

18. The method of claim 1, wherein the culturing step comprises culturing the tissue within a physical barrier on the egg, wherein the barrier comprises an aperture allowing exposure of the tissue to the egg.

19. The method of claim 18, wherein the barrier is ring-shaped.

20. The method of claim 18, wherein the barrier is comprised of biologically inert material.

21. The method of claim 20, wherein the material comprises silicon-based organic polymers.

22. The method of claim 1, wherein the mammal is a human or mouse.

23. The method of claim 22, wherein the mouse is a mouse patient-derived xenograft model.

24. The method of claim 1, wherein the cultured tissue is further provided to a model.

25. The method of claim 24, wherein the model is an in vivo model.

26. The method of claim 25, wherein the model is a patient-derived xenograft mouse model.

27. The method of claim 1, wherein cells from the cultured tissue are used for generating cell lines.

28. The method of claim 1, wherein cells from the cultured tissue are used for flow cytometry or viral transduction.

29. The method of claim 1, wherein the obtained tissue was subject to freezing temperatures prior to the culturing step.

30. The method of claim 1, wherein cells from the cultured tissue are frozen.

31. The method of claim 1, wherein the culturing steps utilize conditions suitable for three-dimensional tumor growth.

32. The method of claim 26, wherein cells from the cell lines or tissue are genetically engineered.

33. (canceled)

34. (canceled)

35. The method of claim 1, wherein the culturing step comprises providing to the cultured tissue one or more types of immune cells.

36. The method of claim 35, wherein the immune cells are obtained from the individual.

37. The method of claim 35, wherein the immune cells are allogeneic to the individual.

38. The method of claim 1, wherein the CAM model is produced using eggs positioned in a horizontal configuration.

39. The method of claim 38, wherein air inside the egg is removed.

40. The method of claim 1, wherein the CAM model is produced using eggs positioned in a vertical configuration.

41. The method of claim 40, wherein air inside the egg is not removed.

42. The method of claim 1, wherein one or more steps of the method are automated.
